(19) United States
(12) Patent Application Publication
(10) Pub. No.: US 2008/0312306 A1
(43) Pub. Date: Dec. 18, 2008

(54) POLYMORPHS, SOLVATES, AND HYDRATE OF 5-(4'-FLUORO-2'-OXO-1',2'-DIHYDROSPIRO[CYCLOPROPANE-1,3'-INDOL]-5'-YL)-1-METHYL-1H-PYRROLE-2-CARBONITRILE

(75) Inventors: Mahmoud Mirmehrabi, Laval (CA); John Leo Considine, Bridgewater, NJ (US); Yuping Niu, River Vale, NJ (US); Abdolsamad Tadayon, Montreal (CA); Subodh Deshmukh, White Plains, NY (US); Jennifer Q. Liang, Morris Plains, NJ (US); Yanzhong Wu, Bardonia, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/138,852

(22) Filed: Jun. 13, 2008

Related U.S. Application Data
(60) Provisional application No. 60/934,658, filed on Jun. 15, 2007.
(60) Provisional application No. 60/934,909, filed on Jun. 15, 2007.
(60) Provisional application No. 60/934,906, filed on Jun. 15, 2007.
(60) Provisional application No. 60/934,905, filed on Jun. 15, 2007.
(60) Provisional application No. 60/934,901, filed on Jun. 15, 2007.
(60) Provisional application No. 60/934,836, filed on Jun. 15, 2007.
(60) Provisional application No. 60/934,761, filed on Jun. 15, 2007.

(51) Int. Cl. A61K 31/404; C07D 403/02; A61P 15/00; A61P 35/00

(52) U.S. Cl. 514/409; 548/411

(57) ABSTRACT

Novel polymorph Form II and III, solvate Forms I, II, III, and IV, and the hydrate of 5-(4′-fluoro-2′-oxo-1′,2′-dihydrospiro[cyclopropane-1,3′-indol]-5′-yl)-1-methyl-1H-pyrrole-2-carbonitrile are provided, as are methods for preparing the same. Pharmaceutical compositions and kits containing these novel polymorphs, solvates, and hydrate, and combinations thereof are further provided, as are methods of contraception; treating or preventing fibroids, uterine leiomyomata, endometriosis, dysfunctional bleeding, polycystic ovary syndrome, hormone-dependent carcinomas; treating symptoms of premenstrual syndrome and premenstrual dysphoric disorder; providing hormone replacement therapy; stimulating food intake; synchronizing estrus; or treating symptoms of premenstrual syndrome using one or more of these polymorphs, solvates, or hydrate.

### CROSS-REFERENCE TO RELATED APPLICATIONS

This application claims the benefit of the priorities of U.S. Provisional Patent Application No. 60/934,658, filed Jun. 15, 2007; U.S. Provisional Patent Application No. 60/934,909, filed Jun. 15, 2007; U.S. Provisional Patent Application No. 60/934,906, filed Jun. 15, 2007; U.S. Provisional Patent Application No. 60/934,905, filed Jun. 15, 2007; U.S. Provisional Patent Application No. 60/934,901, filed Jun. 15, 2007; U.S. Provisional Patent Application No. 60/934,836, filed Jun. 15, 2007; and U.S. Provisional Patent Application No. 60/934,761, filed Jun. 15, 2007.

### BACKGROUND OF THE INVENTION

This invention relates to polymorphs, solvates, and the hydrate of a modulator of the progesterone receptor, its preparation and utility.

Intracellular receptors (IR) form a class of structurally related gene regulators known as “ligand dependent transcription factors” (Mangelsdorf, D. J. et. al., Cell, 83, 835, 1995). The steroid receptor family is a subset of the IR Family, including the progesterone receptor (PR), estrogen receptor (ER), androgen receptor (AR), glucocorticoid receptor (GR), and mineralocorticoid receptor (MR).

The natural hormone, or ligand, for the PR is the steroid progesterone, but synthetic compounds, such as medroxyprogesterone acetate or levonorgestrel, can also serve as PR ligands. Once a ligand is present in the fluid surrounding a cell, it passes through the membrane via passive diffusion, and binds to the IR to create a receptor/ligand complex. This complex binds to specific gene promoters present in the cell's DNA. Once bound to the DNA the complex modulates the production of mRNA and the protein encoded by that gene.

A compound that binds to an IR and mimics the action of the natural hormone is termed an agonist, whilst a compound which inhibits the effect of the hormone is an antagonist.

PR agonists (natural and synthetic) are known to play an important role in the health of women. PR agonists are used in birth control formulations, either alone or in the presence of an ER agonist.

PR antagonists may also be used in contraception (Ulmann, et al., Ann. N.Y. Acad. Sci., 261, 248, 1995; Kekkonen, et al, Fertility and Sterility, 60, 610, 1993; or U.S. Pat. No. 5,719,136); for the treatment of hormone dependent breast cancers (Horwitz, et al, Horm. Cancer, 283, 1996, pub: Birkhaeuser, Boston, Mass., ed. Vedeckis), uterine and ovarian cancers, non-malignant chronic conditions such as uterine fibroids (Murphy, et al, J. Clin. Endo. Metab., 76, 513, 1993) and endometriosis (Kettel, et al., Fertility and Sterility, 56, 402, 1991); the treatment of hormone dependent prostate cancer (Michna, et al, Ann. N.Y. Acad. Sci., 761, 224, 1995); and for hormone replacement therapy (U.S. Pat. No. 5,719,136).

5-(4′-fluoro-2′-=oxo-1′,2′-dihydrospiro[cyclopropane-1,3′-indol]-5′-yl)-1-methyl-1H-pyrrole-2-carbonitrile polymorph Form I has been described in US Patent Application Publication Nos. US-2006/0030717 and US-2008/0051585, which are hereby incorporated by reference, and is a progesterone receptor modulator.

What is needed are alternate forms of 5-(4′-fluoro-2′-oxo-1′,2′-dihydrospiro[cyclopropane-1,3′-indol]-5′-yl)-1-methyl-1H-pyrrole-2-carbonitrile.

### SUMMARY OF THE INVENTION

In one aspect, polymorph Forms II and III, solvate Forms I, II, III, and IV, and the hydrate of 5-(4′-fluoro-2′-oxo-1′,2′-dihydrospiro[cyclopropane-1,3′-indol]-5′-yl)-1-methyl-1H-pyrrole-2-carbonitrile are provided.

In another aspect, methods for preparing polymorph Forms II and III, solvate Forms I, II, III, and IV, and the hydrate of 5-(4′-fluoro-2′-oxo-1′,2′-dihydrospiro[cyclopropane-1,3′-indol]-5′-yl)-1-methyl-1H-pyrrole-2-carbonitrile are provided.

In a further aspect, pharmaceutical compositions containing polymorph Forms II and/or III, solvate Forms I, II, III, and/or IV, and/or the hydrate of 5-(4′-fluoro-2′-oxo-1′,2′-dihydrospiro[cyclopropane-1,3′-indol]-5′-yl)-1-methyl-1H-pyrrole-2-carbonitrile and a pharmaceutically acceptable carrier are provided.

In yet another aspect, kits containing polymorph Forms II and/or III, solvate Forms I, II, III, and/or IV, and/or the hydrate of 5-(4′-fluoro-2′-oxo-1′,2′-dihydrospiro[cyclopropane-1,3′-indol]-5′-yl)-1-methyl-1H-pyrrole-2-carbonitrile are provided.

In still a further aspect, methods of contraception; treating or preventing fibroids, uterine leiomyomata, endometriosis, dysfunctional bleeding, polycystic ovary syndrome, or hormone-dependent carcinomas; providing hormone replacement therapy; stimulating food intake; synchronizing estrus; or treating symptoms of premenstrual syndrome or premenstrual dysphoric disorder are provided and include administering to a mammal in need thereof a pharmaceutically effective amount of polymorph Forms TI and/or III, solvate Forms I, II, III, and/or IV, and/or the hydrate of 5-(4′-fluoro-2′-oxo-1′,2′-dihydrospiro[cyclopropane-1,3′-indol]-5′-yl)-1-methyl-1H-pyrrole-2-carbonitrile.

Other aspects and advantages of the present invention are described further in the following detailed description of the preferred embodiments thereof.

### BRIEF DESCRIPTION OF THE DRAWINGS

FIG. 1 provides the x-ray diffraction (XRD) pattern for a sample of the hydrate of 5-(4′-fluoro-2′-oxo-1′,2′-dihydrospiro[cyclopropane-1,3′-indol]-5′-yl)-1-methyl-1H-pyrrole-2-carbonitrile.

FIG. 2 provides the differential scanning calorimetry (DSC) thermogram for a sample of the hydrate of 5-(4′-fluoro-2′-oxo-1′,2′-dihydrospiro[cyclopropane-1,3′-indol]-5′-yl)-1-methyl-1H-pyrrole-2-carbonitrile.

FIG. 3 provides the thermograviometric analysis (TGA) curve for a sample of the hydrate of 5-(4′-fluoro-2′-oxo-1′,2′-dihydrospiro[cyclopropane-1,3′-indol]-5′-yl)-1-methyl-1H-pyrrole-2-carbonitrile with 3.9% water loss.

FIG. 4 provides the XRD pattern for a sample of polymorph Form II of 5-(4′-fluoro-2′-oxo-1′,2′-dihydrospiro[cyclopropane-1,3′-indol]-5′-yl)-1-methyl-1H-pyrrole-2-carbonitrile.

FIG. 5 provides the DSC thermogram for a sample of polymorph Form II of 5-(4′-fluoro-2′-oxo-1′,2′-dihydrospiro[cyclopropane-1,3′-indol]-5′-yl)-1-methyl-1H-pyrrole-2-carbonitrile.

FIG. 6 provides the XRD pattern for a sample of polymorph Form III of 5-(4′-fluoro-2′-oxo-1′,2′-dihydrospiro[cyclopropane-1,3′-indol]-5′-yl)-1-methyl-1H-pyrrole-2-carbonitrile.

FIG. 7 provides the DSC thermogram for a sample of polymorph Form III of 5-(4′-fluoro-2′-oxo-1′,2′-dihydrospiro[cyclopropane-1,3′-indol]-5′-yl)-1-methyl-1H-pyrrole-2-carbonitrile.

FIG. 8 provides the XRD pattern For a sample of solvate Form I of 5-(4′-fluoro-2′-oxo-1′,2′-dihydrospiro[cyclopropane-1,3′-indol]-5′-yl)-1-methyl-1H-pyrrole-2-carbonitrile.

FIG. 9 provides the XRD pattern for a sample of solvate Form II of 5-(4′-fluoro-2′-oxo-1′,2′-dihydrospiro[cyclopropane-1,3′-indol]-5′-yl)-1-methyl-1H-pyrrole-2-carbonitrile.

FIG. 10 provides the DSC thermogram for a sample of solvate Form II of 5-(4′-fluoro-2′-oxo-1′,2′-dihydrospiro[cyclopropane-1,3′-indol]-5′-yl)-1-methyl-1H-pyrrole-2-carbonitrile.

FIG. 11 provides the XRD pattern for a sample of solvate Form III of 5-(4′-fluoro-2′-oxo-1′,2′-dihydrospiro[cyclopropane-1,3′-indol]-5′-yl)-1-methyl-1H-pyrrole-2-carbonitrile.

FIG. 12 provides the DSC thermogram for a sample of solvate Form III of 5-(4′-fluoro-2′-oxo-1′,2′-dihydrospiro[cyclopropane-1,3′-indol]-5′-yl)-1-methyl-1H-pyrrole-2-carbonitrile.

FIG. 13 provides the XRD pattern for a sample of solvate Form IV of 5-(4′-fluoro-2′-oxo-1′,2′-dihydrospiro[cyclopropane-1,3′-indol]-5′-yl)-1-methyl-1H-pyrrole-2-carbonitrile.

FIG. 14 provides the nuclear magnetic resonance (NMR) spectrum for a sample of solvate Form IV of 5-(4′-fluoro-2′-oxo-1′,2′-dihydrospiro[cyclopropane-1,3′-indol]-5′-yl)-1-methyl-1H-pyrrole-2-carbonitrile.

### DETAILED DESCRIPTION OF THE INVENTION

Novel polymorphs, solvates, and the hydrate of 5-(4′-fluoro-2′-oxo-1′,2′-dihydrospiro[cyclopropane-1,3′-indol]-5′-yl)-1-methyl-1H-pyrrole-2-carbonitrile, denoted herein as polymorph Form II, polymorph Form III, solvate Form I, solvate Form II, solvate Form III, solvate Form IV, and the hydrate are described. The polymorphs, solvates and hydrates differ from each other, e.g., physically and/or chemically, and polymorph Form I described in the art in the structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile, as determined by spectroscopic techniques such as XRD, DSC, NMR, TGA, among others.

As used herein, “Form I” or “Polymorph Form I” are used interchangeably and refer to 5-(4′-fluoro-2′-oxo-1′,2′-dihydrospiro[cyclopropane-1,3′-indol]-5′-yl)-1-methyl-1H-pyrrole-2-carbonitrile. 5-(4′-fluoro-2′-oxo-1′,2′-dihydrospiro[cyclopropane-1,3′-indol]-5′-yl)-1-methyl-1H-pyrrole-2-carbonitrile can prepared according to the procedure set forth in US Patent Application Publication No. US-2008/0051585, which is hereby incorporated by reference. Polymorph Form I of 5-(4′-fluoro-2′-oxo-1′,2′-dihydrospiro[cyclopropane-1,3′-indol]-5′-yl)-1-methyl-1H-pyrrole-2-carbonitrile contains a peak at 2θ at about 18.5° at greater than about 95% relative intensity. Polymorph Form I of 5-(4′-fluoro-2′-oxo-1′,2′-dihydrospiro[cyclopropane-1,3′-indol]-5′-yl)-1-methyl-1H-pyrrole-2-carbonitrile also contains an endothermic peak with a Tₒₙₛₑₜ of about 223° C., as determined by DSC.

“Form I” and “Polymorph Form I” also refer to both non-micronized and micronized forms of the same. Micronization of polymorph Form I of 5-(4′-fluoro-2′-oxo-1′,2′-dihydrospiro[cyclopropane-1,3′-indol]-5′-yl)-1-methyl-1H-pyrrole-2-carbonitrile is typically accomplished under nitrogen and conventional micronizing techniques, e.g., with a Trost or jet mill, applied to non-micronized 5-(4′-fluoro-2′-oxo-1′,2′-dihydrospiro[cyclopropane-1,3′-indol]-5′-yl)-1-methyl-1H-pyrrole-2-carbonitrile. Micronized 5-(4′-fluoro-2′-oxo-1′,2′-dihydrospiro[cyclopropane-1,3′-indol]-5′-yl)-1-methyl-1H-pyrrole-2-carbonitrile polymorph Form I has median particle size less than about 8 μm±1 μm.

The inventors found several advantages of these new forms of 5-(4′-fluoro-2′-oxo-1′,2′-dihydrospiro[cyclopropane-1,3′-indol]-5′-yl)-1-methyl-1H-pyrrole-2-carbonitrile. In one embodiment, polymorph Form II of 5-(4′-fluoro-2′-oxo-1′,2′-dihydrospiro[cyclopropane-1,3′-indol]-5′-yl)-1-methyl-1H-pyrrole-2-carbonitrile has the advantage, over the hydrate of 5-(4′-fluoro-2′-oxo-1′,2′-dihydrospiro[cyclopropane-1,3′-indol]-5′-yl)-1-methyl-1H-pyrrole-2-carbonitrile, in that it is not hygroscopic. In a further embodiment, polymorph Form II is more thermodynamically stable over polymorph Form I and does not convert to other polymorphs if slurried in a solvent. In another embodiment, polymorph Form III of 5-(4′-fluoro-2′-oxo-1′,2′-dihydrospiro[cyclopropane-1,3′-indol]-5′-yl)-1-methyl-1H-pyrrole-2-carbonitrile is more soluble than other crystallographic forms of 5-(4′-fluoro-2′-oxo-1′,2′-dihydrospiro[cyclopropane-1,3′-indol]-5′-yl)-1-methyl-1H-pyrrole-2-carbonitrile. In still a further embodiment, polymorph Form III is more soluble than polymorph Form II. In yet a further embodiment, samples of the hydrate of 5-(4′-fluoro-2′-oxo-1′,2′-dihydrospiro[cyclopropane-1,3′-indol]-5′-yl)-1-methyl-1H-pyrrole-2-carbonitrile were hygroscopic.

Characterization of the novel polymorphs, solvates and hydrate and distinguishing the same from polymorph Form I and each other is accomplished using techniques known to those of skill in the art. Specifically, verification that the same are present can be performed using techniques including melting point, infrared (IR) spectroscopy, NMR spectroscopy, mass spectral (MS) analysis, combustion analysis, Raman spectroscopy, elemental analysis, chromatography including high performance liquid chromatography, TGA, and microscopy. Other techniques including DSC and XRD are also useful in distinguishing these novel polymorphs, hydrates, and solvates from polymorph Form I.

In one embodiment, XRD techniques are utilized to distinguish the novel polymorphs, solvates, and hydrate from polymorph Form I. Using the teachings herein, one of skill in the art would readily be able to determine the conditions required to obtain an XRD pattern of the novel polymorphs, solvates, hydrate and polymorph Form I. A variety of XRD instruments are available and include the D8 ADVANCE® X-ray powder diffractometer (Bruker), among others.

In another embodiment, DSC techniques are utilized to distinguish the novel polymorphs, solvates, and hydrate of 5-(4′-fluoro-2′-oxo-1′,2′-dihydrospiro[cyclopropane-1,3′-indol]-5′-yl)-1-methyl-1H-pyrrole-2-carbonitrile from polymorph Form I. One of skill in the art would readily be able to determine the conditions necessary to obtain DSC thermograms of the novel polymorphs, solvates, and hydrate. A variety of differential scanning calorimeters are available to those of skill in the art and include the Q SERIES™ DSC Q1000 instrument (TA instruments) using temperatures of about 25° C. to about 230° C. and temperature increases at various rates including 5° C./minute, 10° C./minute, and 30° C./minute, among other instruments and conditions.

In a further embodiment, solid state nuclear magnetic resonance (NMR) is utilized to distinguish the novel polymorphs, solvates, and hydrate of 5-(4′-fluoro-2′-oxo-1′,2′-dihydrospiro[cyclopropane-1,3′-indol]-5′-yl)-1-methyl-1H-pyrrole-2-carbonitrile from polymorph Form I. One of skill in the art would readily be able to determine the conditions necessary to obtain a solid state NMR spectrum. A variety of NMR instruments useful for solid state NMR are available and could readily be selected by those of skill in the art. Solution NMR can also be used to verify the purity and presence of the novel polymorphs, solvates, and/or hydrate of 5-(4′-fluoro-2′-oxo-1′,2′-dihydrospiro[cyclopropane-1,3′-indol]-5′-yl)-1-methyl-1H-pyrrole-2-carbonitrile. A variety of NMR instruments for solution NMR is available to and could readily be selected by those of skill in the art, as could the frequency of the NMR instrument. One of skill in the art would also be able to readily select a suitable solvent, including isotope labeled solvents such as ²H or ¹³C labeled solvents. In one embodiment, NMR spectra were acquired on a BRUKER® 300 MHz spectrometer using a deuterated solvent and tetramethylsilane (TMS) as an internal standard (δ=0.0 ppm). All 6 values are expressed in units of parts per million (ppm).

In another embodiment, microscopy may be utilized to determine the crystallographic shape of the particles of the novel polymorphs, solvates, and hydrate. One of skill in the art would readily be able to select a suitable microscope for such an analysis and may include microscopes obtained from Leica.

### A. The Hydrate of 5-(4′-fluoro-2′-oxo-1′,2′-dihydrospiro[cyclopropane-1,3′-indol]-5′-yl)-1-methyl-1H-pyrrole-2-carbonitrile

Provided herein is the hydrate of 5-(4′-fluoro-2′-oxo-1′,2′-dihydrospiro[cyclopropane-1,3′-indol]-5′-yl)-1-methyl-1H-pyrrole-2-carbonitrile and methods for preparing the same. The hydrate has a different structure of the crystal lattice of 5-(4′-fluoro-2′-oxo-1′,2′-dihydrospiro[cyclopropane-1,3′-indol]-5′-yl)-1-meth-yl-1H-pyrrole-2-carbonitrile from polymorph Form I described in the art and the novel polymorph Forms II and III and solvate Forms I, II, III, and IV described herein

In one embodiment, the hydrate is prepared from polymorph Form I. This method includes slurrying polymorph Form I in water at about room temperature. In one example, polymorph Form I is slurried in water for more than about 1 week. The hydrate is desirably isolated from the solution using filtration. However, other techniques may be utilized as determined by one of skill in the art. Once collected, the hydrate is dried, typically under reduced pressures, such as under a full vacuum. In another example, Form I is slurried in water for 1 to 5 days. In another example, Form I is slurried in water for at least about 60 hours. In still a further example, Form I is slurried in water for about 1 to about 2 weeks. The hydrate is desirably isolated from the solution using techniques known to those of skill in the art. Desirably, crystallization and filtration are utilized to collect the hydrate. However, other techniques may be utilized as determined by one of skill in the art. Once collected, the hydrate is dried, typically under reduced pressures. In one embodiment, the hydrate may be dried at about 40 to about 45° C. typically under reduced pressures, e.g., about 30 to about 50 mm Hg.

As used herein, the term “slurrying” describes the technique of mixing, i.e., stirring, a solid in a liquid which is not a solvent for the solid or in which the solid is only partially soluble, such that a suspension is formed. The mixing may be performed at various speeds and temperatures and for varying amounts of time. Desirably, the slurrying is performed using an apparatus which stirs the suspension. However, the physical task of performing the slurrying does not limit the present application.

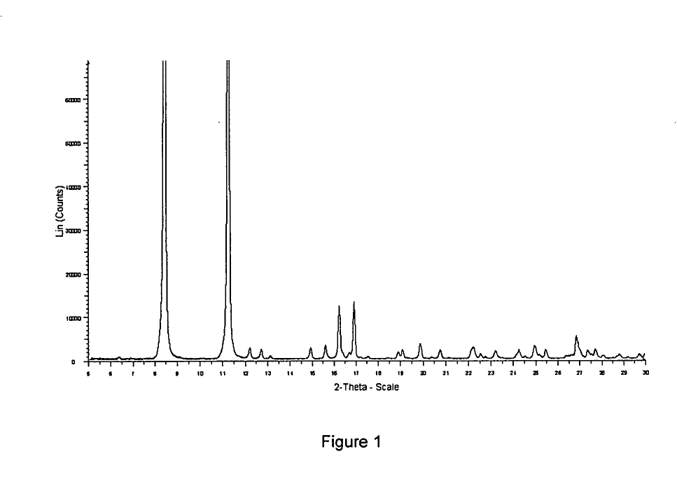

Characterization of the hydrate and distinguishing the same from polymorph Form I and polymorph Forms II and III and solvate Forms I, II, III, and IV is accomplished using techniques discussed above. The powder XRD pattern of the hydrate described herein was obtained using X-ray crystallographic techniques known to those of skill in the art. See, FIG. 1. In one embodiment, the XRD pattern of the hydrate includes multiple peaks that differ from the XRD peaks obtained for polymorph Forms I, II, or III and solvate Forms I, II, III, and IV. In another embodiment, the XRD pattern of the hydrate contains one large peak and several smaller peaks. The XRD for the hydrate includes peaks at 2θ of about 8.4°±0.3° at greater than about 95% relative intensity. Desirably, the peak at 2θ of about 8.4°±0.3° is at about 100% relative intensity. The XRD for the hydrate may also include peaks at 2θ of about 11.2°±0.3° and 16.9°±0.3° at varying intensities. Desirably, the XRD for the hydrate lacks the peak for polymorph Form I at about 18.5°.

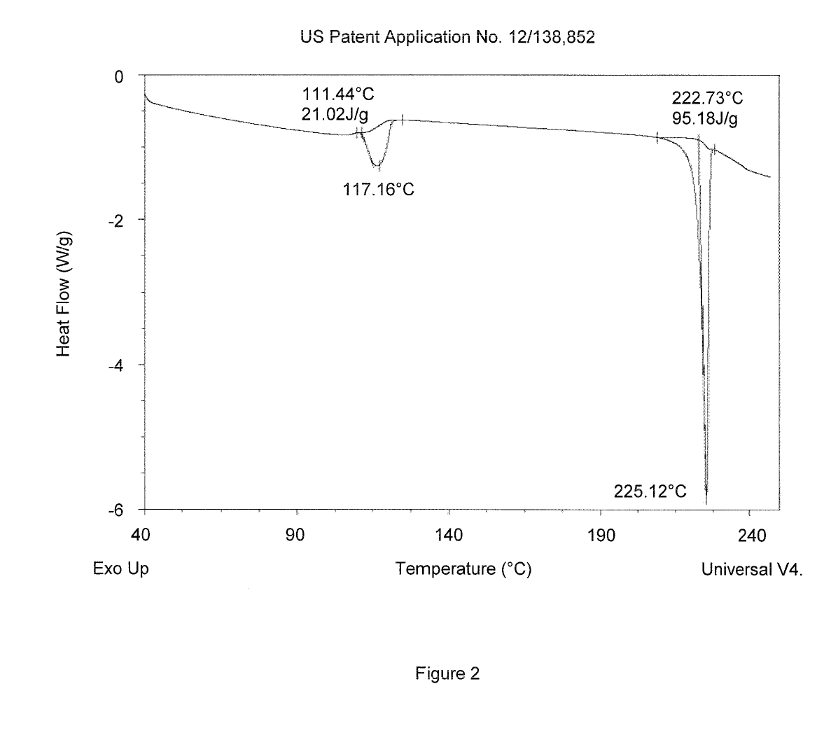

DSC techniques were utilized to distinguish the hydrate of 5-(4′-fluoro-2′-oxo-1′,2′-dihydrospiro[cyclopropane-1,3′-indol]-5′-yl)-1-methyl-1H-pyrrole-2-carbonitrile from polymorph Form I. See, FIG. 2. In one embodiment, the DSC thermogram of the hydrate differs from the DSC thermogram of polymorph Form I and includes a first endothermic peak with a Tₒₙₛₑₜ of about 223° C. and a second endothermic peak with a Tₒₙₛₑₜ of about 111° C.

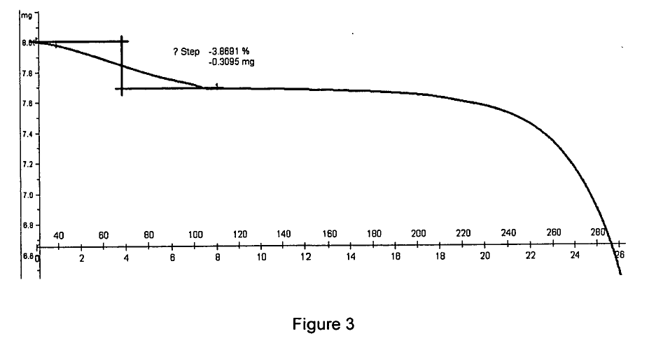

Other techniques may be utilized to characterize the hydrate. In one embodiment, solution NMR is used to verify the purity and presence of the hydrate of 5-(4′-fluoro-2′-oxo-1′,2′-dihydrospiro[cyclopropane-1,3′-indol]-5′-yl)-1-methyl-1H-pyrrole-2-carbonitrile and specifically the presence of water in the lattice. In another embodiment, TGA data is collected and shown in FIG. 3. This TGA data further supports the presence of the hydrate in view of the 3.9% water loss.

The hydrate has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from the Form I described in the art as determined by XRD and DSC. Specifically, the hydrate contains water in the crystal lattice. Specifically, as evidenced by dynamic vapor sorption (DVS), the hydrate contains about 2.5% to about 5% water. In one embodiment, the hydrate contains about 2.5% water at 0% relative humidity (RH). In another embodiment, the hydrate contains about 4.75% water at 100% RH.

The hydrate of 5-(4′-fluoro-2′-oxo-1′,2′-dihydrospiro[cyclopropane-1,3′-indol]-5′-yl)-1-methyl-1H-pyrrole-2-carbonitrile is also stable under a variety of conditions. In one example, the hydrate is stable in water. In another example, the hydrate is stable under conditions of micronization. In a further example, the hydrate is stable under a vacuum for a period of at least about 48 hours.

The hydrate is also useful in preparing polymorphs, including polymorph Forms I, II, and III and solvate Forms I, II, III, and IV, as discussed below. In one embodiment, the hydrate of 5-(4′-fluoro-2′-oxo-1′,2′-dihydrospiro[cyclopropane-1,3′-indol]-5′-yl)-1-methyl-1H-pyrrole-2-carbonitrile may be utilized to prepare polymorph Form I of 5-(4′-fluoro-2′-oxo-1′,2′-dihydrospiro[cyclopropane-1,3′-indol]-5′-yl)-1-methyl-1H-pyrrole-2-carbonitrile. The method includes slurrying the hydrate in a solvent comprising ethyl acetate, methanol, or isopropylalcohol (IPA) at room temperature for about 48 to about 60 hours. Polymorph Form I may then be isolated using filtration.

In a further embodiment, the hydrate may be utilized to prepare polymorph Form II of 5-(4′-fluoro-2′-oxo-1′,2′-dihydrospiro[cyclopropane-1,3′-indol]-5′-yl)-1-methyl-1H-pyrrole-2-carbonitrile. The method includes heating the hydrate to a temperature of about 125 to about 130° C. which results in solid polymorph Form II.

In another embodiment, the hydrate may be utilized to prepare polymorph Form II of 5-(4′-fluoro-2′-oxo-1′,2′-dihydrospiro[cyclopropane-1,3′-indol]-5′-yl)-1-methyl-1H-pyrrole-2-carbonitrile comprising heating the hydrate to at least about 50° C. under full vacuum. The polymorph Form II is thereby isolated as a solid. In one example, the temperature is about 50° C. to about 130° C. in another example, the temperature is about 50 to about 60° C. Desirably, the heating is performed for at least about 48 hours.

In a further embodiment, the hydrate may be utilized to prepare polymorph Form III of 5-(4′-fluoro-2′-oxo-1′,2′-dihydrospiro[cyclopropane-1,3′-indol]-5′-yl)-1-methyl-1H-pyrrole-2-carbonitrile. The method includes dissolving the hydrate in acetone. The acetone solution is then heated to a temperature of about 45 to about 55° C. After heating, the acetone is evaporated from the solution using techniques known to those of skill in the a to provide dry Form III.

In a further embodiment, the hydrate may be utilized to prepare solvate Form I of 5-(4′-fluoro-2′-oxo-1′,2′-dihydrospiro[cyclopropane-1,3′-indol]-5′-yl)-1-methyl-1H-pyrrole-2-carbonitrile. The method includes slurrying the hydrate in dimethylformamide (DMF) at about room temperature. In one embodiment, the hydrate is slurried in DMF for at least about 60 hours. In another embodiment, the hydrate is slurried in DMF for about 60 hours to about 7 days. Desirably, the slurrying is performed at about room temperature. Solvate Form I may then be isolated from the solution using techniques known to those of skill in the art. Desirably, filtration and decanting is utilized to collect solvate Form I. However, other techniques may be utilized as determined by one of skill in the art.

In yet another embodiment, the hydrate may be utilized to prepare solvate Form II of 5-(4′-fluoro-2′-oxo-1′,2′-dihydrospiro[cyclopropane-1,3′-indol]-5′-yl)-1-methyl-1H-pyrrole-2-carbonitrile. The method includes dissolving the hydrate in dimethylformamide (DMF). The DMF solution is then heated to about 45 to about 55° C. The DMF is then evaporated from the solution using full vacuum to obtain a solid solvate Form II.

In still a further embodiment, the hydrate may be utilized to prepared solvate Form III of 5-(4′-fluoro-2′-oxo-1′,2′-dihydrospiro[cyclopropane-1,3′-indol]-5′-yl)-1-methyl-1H-pyrrole-2-carbonitrile. The method includes slurrying the hydrate in toluene. In one embodiment, the hydrate is slurried in toluene for at least about 60 hours. In another embodiment, the hydrate is slurried in toluene for about 60 hours to about 7 days. Solvate Form III may then be isolated from the solution using filtration. However, other techniques may be utilized for isolation as determined by one of skill in the art.

In still another embodiment, the hydrate may be utilized to prepare solvate Form IV of 5-(4′-fluoro-2′-oxo-1′,2′-dihydrospiro[cyclopropane-1,3′-indol]-5′-yl)-1-methyl-1H-pyrrole-2-carbonitrile. The method includes dissolving the hydrate in tetrahydrofuran (THF). Heptane is then quickly added to the THF solution. The term “quickly” as used herein describes the rate of addition of the heptane. Desirably, the heptane is poured into the THF solution without stopping. Desirably, the THF solution is stirred during heptane addition. In one embodiment, excess heptane is added to the THF solution. In another embodiment, the volume of THF to heptane is 1:1. The amount of “excess” heptane could readily be determined by one of skill in the art. In one embodiment, the term excess refers to a heptane volume that is 1.5 times or greater the amount of THF. In another embodiment, excess refers to a volume that is 1.5, 2, 3, 4, 5, 6, 7, 8, 9, or 10 times the amount of THF. Solvate Form IV may then be isolated from the solution using techniques known to those of skill in the art. Desirably, filtration is utilized to collect solvate Form IV. However, other techniques may be utilized as determined by one of skill in the art.

### B. Polymorph Form II of 5-(4′-fluoro-2′-oxo-1′,2′-dihydrospiro[cyclopropane-1,3′-indol]-5′-yl)-1-methyl-1H-pyrrole-2-carbonitrile

Also provided herein is a novel polymorph of 5-(4′-fluoro-2′-oxo-1′,2′-dihydrospiro[cyclopropane-1,3′-indol]-5′-yl)-1-methyl-1H-pyrrole-2-carbonitrile, designated as polymorph Form II, and methods for preparing this novel polymorph. Polymorph Form II has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and novel polymorph Form III, solvate Forms I, II, III, and IV, and the hydrate described herein

In one embodiment, polymorph Form II of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. The hydrate of 5-(4′-fluoro-2′-oxo-1′,2′-dihydrospiro[cyclopropane-1,3′-indol]-5′-yl)-1-methyl-1H-pyrrole-2-carbonitrile is thereby slurried in a solvent such as ethyl acetate, methanol, or isopropyl alcohol. Typically, the hydrate is slurried in the selected solvent at room temperature for about 60 hours to about 7 days.

In a further embodiment, the hydrate is heated to a temperature of about 125 to about 130° C. which results in solid polymorph Form II.

In another embodiment, the hydrate is heated to at least about 50° C. under full vacuum. The polymorph Form II is thereby isolated as a solid. In one example, the temperature is about 50° C. to about 130° C. In another example, the temperature is about 50 to about 60° C. Desirably, the heating is performed for at least about 48 hours.

In another embodiment, polymorph Form II is prepared from polymorph Form I. Polymorph Form I of 5-(4′-fluoro-2′-oxo-1′,2′-dihydrospiro[cyclopropane-1,3′-indol]-5′-yl)-1-methyl-1H-pyrrole-2-carbonitrile is slurried in a solvent such as ethyl acetate, methanol, or isopropyl alcohol. Typically, the hydrate is slurried in the respective solvent at room temperature for about 60 hours to about 7 days.

Regardless of whether polymorph Form II is prepared from polymorph Form I or the hydrate, it is isolated from the solution using techniques known to those of skill in the art. Desirably, filtration and decanting is utilized to collect polymorph Form II. However, other techniques may be utilized as determined by one of skill in the art. Once collected, polymorph Form II is dried, typically under reduced pressures, such as under a full vacuum.

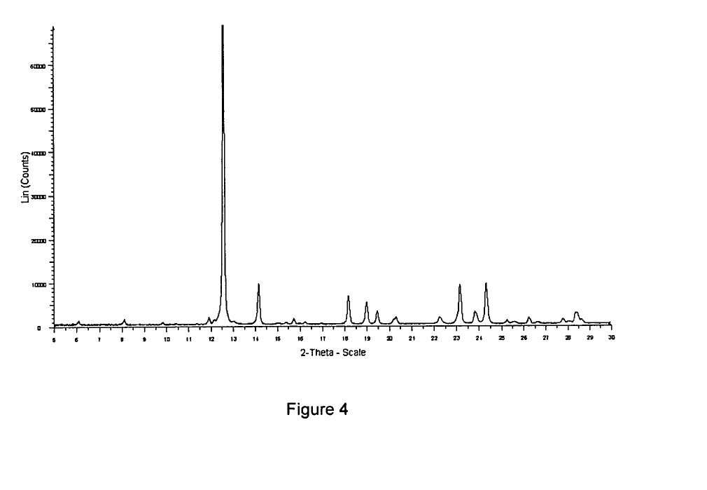

The powder XRD pattern of polymorph Form II described herein was obtained using X-ray crystallographic techniques known to those of skill in the art. See, FIG. 4.

In one embodiment, the XRD pattern of polymorph Form II includes multiple peaks that differ from the XRD peaks obtained for polymorph Forms I or III, solvate Forms I, II, III, and IV, and the hydrate. In another embodiment, the XRD pattern of polymorph Form II contains one large peak and several smaller peaks. The XRD for polymorph Form II includes peaks at 2θ of about 12.50±0.30 at greater than about 95% relative intensity. Desirably, the peak at 2θ of about 12.50±0.3° is at about 100% relative intensity. The XRD for polymorph Form II may also include peaks at 2θ of about 14.1°±0.3°, 18.9°±0.30, and 24.30±0.30 at varying intensities. Desirably, the XRD for polymorph Form II lacks the peak for polymorph Form I at about 18.5°.

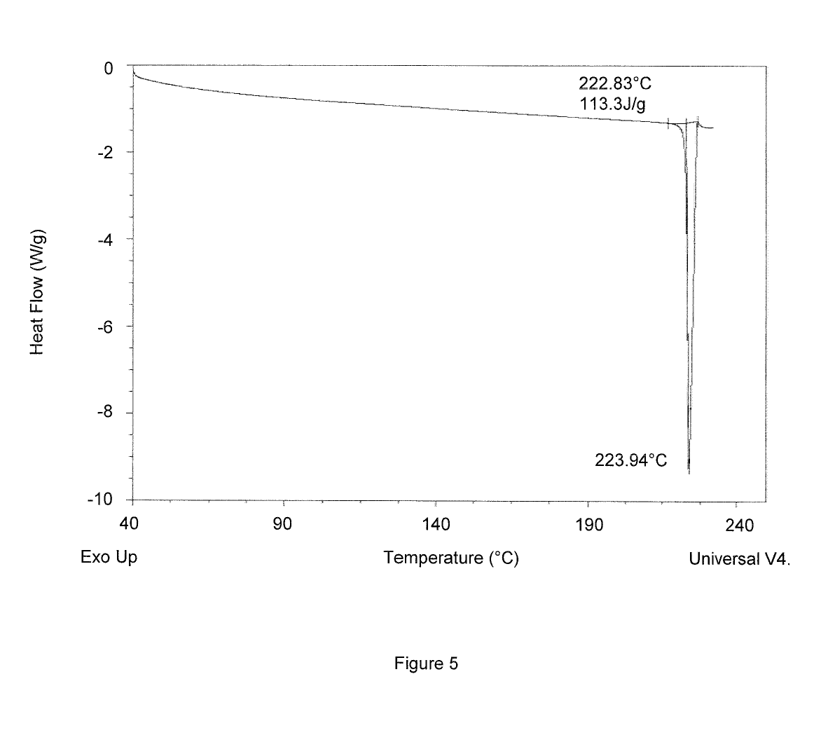

DSC techniques can also be utilized to distinguish polymorph Form II of 5-(4′-fluoro-2′-oxo-1′,2′-dihydrospiro[cyclopropane-1,3′-indol]-5′-yl)-1-methyl-1H-pyrrole-2-carbonitrile from polymorph Form I. In one embodiment, the DSC thermogram of polymorph Form II differs from the DSC thermogram of polymorph Form I and includes an endothermic peak with a Tₒₙₛₑₜ of about 223° C.±1° C. See, FIG. 5.

Microscopy may further be utilized to determine the crystallographic shape of the particles of polymorph Form II. Microscopy at a magnification of about 200× illustrated that the majority of particles obtained from a sample of polymorph Form II were short, rod-like particles.

### C. Polymorph Form III of 5-(4′-fluoro-2′-oxo-1′,2′-dihydrospiro[cyclopropane-1,3′-indol]-5′-yl)-1-methyl-1H-pyrrole-2=carbonitrile

A second novel polymorph, i.e., polymorph Form III, of 5-(4′-fluoro-2′-oxo-1′,2′-dihydrospiro[cyclopropane-1,3′-indol]-5′-yl)-1-methyl-1H-pyrrole-2-carbonitrile, and method for preparing the same are provided. Polymorph Form III has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and the novel polymorph Form II, solvate Forms I, II, III, and IV, and the hydrate described herein

In one embodiment, polymorph Form III of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. Specifically, polymorph Form III of 5-(4′-fluoro-2′-oxo-1′,2′-dihydrospiro[cyclopropane-1,3′-indol]-5′-yl)-1-methyl-1H-pyrrole-2-carbonitrile is prepared by dissolving the hydrate of 5-(4′-fluoro-2′-oxo-1′,2′-dihydrospiro[cyclopropane-1,3′-indol]-5′-yl)-1-methyl-1H-pyrrole-2-carbonitrile in acetone. The acetone solution is then heated to a temperature of about 40° C. to about 55° C. overnight. In one example, the acetone solution is heated to a temperature of about 45 to about 55° C. After heating, the acetone is evaporated from the solution using techniques known to those of skill in the art to provide polymorph Form III.

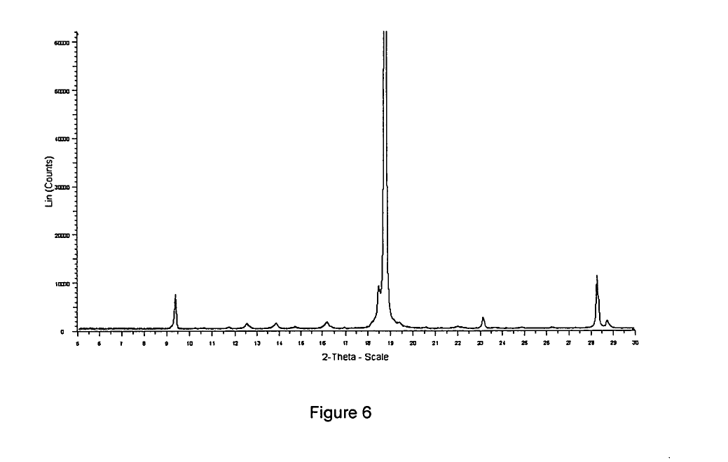

The powder XRD pattern of polymorph Form III described herein was obtained using X-ray crystallographic techniques known to those of skill in the art. See, FIG. 6. In one embodiment, the XRD pattern of polymorph Form III includes multiple peaks that differ from the XRD peaks obtained for polymorph Forms I and II, solvate Forms I, II, III, and IV, and the hydrate. In another embodiment, the XRD pattern of polymorph Form III contains one large peak and several smaller peaks. The XRD for polymorph Form III includes peaks at 2θ of about 18.70±0.30 at greater than about 95% relative intensity. Desirably, the peak at 2θ of about 18.70±0.30 is at about 100% relative intensity. The XRD for polymorph Form III may also include peaks at 2θ of about 9.4°±0.3°, 18.7°±0.3°, and 28.7±0.3° at varying intensities. Desirably, the XRD for polymorph Form III lacks the peak for polymorph Form I at about 18.5°.

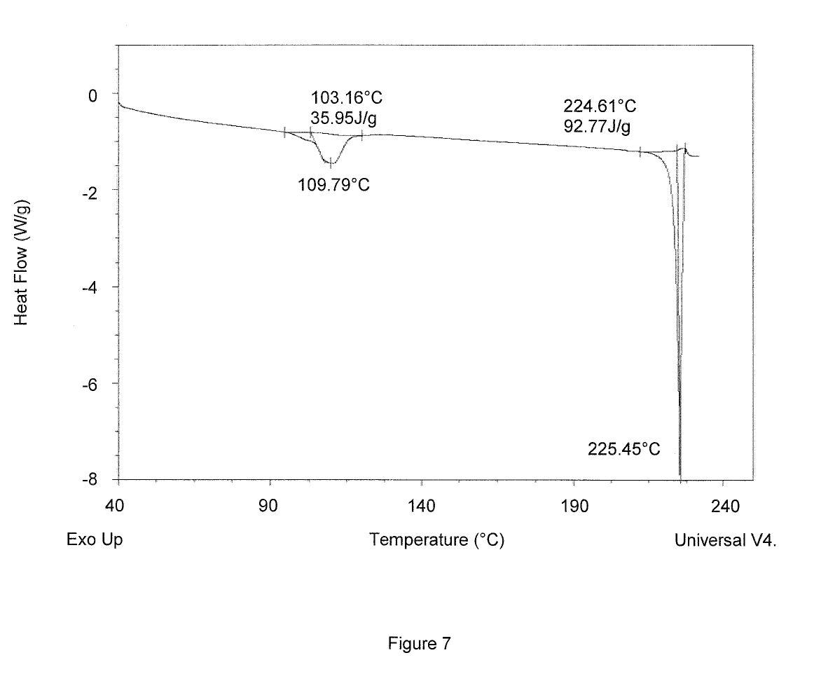

DSC techniques can also be utilized to distinguish polymorph Form III of 5-(4′-fluoro-2′-oxo-1′,2′-dihydrospiro[cyclopropane-1,3′-indol]-5′-yl)-1-methyl-1H-pyrrole-2-carbonitrile from polymorph Form I. See, FIG. 7. In one embodiment, the DSC thermogram of polymorph Form III differs from the DSC thermogram of polymorph Form I and includes an endothermic peak with a Tₒₙₛₑₜ of about 220.5° C.±1° C. In another embodiment, the DSC thermogram of polymorph Form III differs from the DSC thermogram of polymorph Form I, i.e., it does not include an endothermic peak with a Tₒₙₛₑₜ of about 223° C.

### D. Solvate Form I of 5-(4′-fluoro-2′-oxo-1′,2′-dihydrospiro[cyclopropane-1,3′-indol]-5′-yl)-1-methyl-1H-pyrrole-2-carbonitrile

In addition to novel polymorphs, the inventors prepared novel solvates of 5-(4′-fluoro-2′-oxo-1′,2′-dihydrospiro[cyclopropane-1,3′-indol]-5′-yl)-1-methyl-1H-pyrrole-2-carbonitrile. In one embodiment, solvate Form I of 5-(4′-fluoro-2′-oxo-1′,2′-dihydrospiro[cyclopropane-1,3′-indol]-5′-yl)-1-methyl-1H-pyrrole-2-carbonitrile is provided herein, as are methods for preparing solvate Form I. Solvate Form I has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and the novel polymorph Forms II and iii, solvate Forms II, III, and IV, and the hydrate described herein as determined by XRD and DSC. Specifically, as evidenced by spectroscopic techniques, solvate Form I contains dimethylformamide in the crystal lattice.

In one embodiment, solvate Form I of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. Specifically, solvate Form I of 5-(4′-fluoro-2′-oxo-1′,2′-dihydrospiro[cyclopropane-1,3′-indol]-5′-yl)-1-methyl-1H-pyrrole-2-carbonitrile is prepared by slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in dimethylformamide (DMF). In one embodiment, the hydrate is slurried in DMF for at least about 60 hours. In another embodiment, the hydrate is slurried in DMF for about 60 hours to about 7 days. Desirably, the slurrying is performed at about room temperature. Solvate Form I may then be isolated from the solution using filtration. However, other techniques, such as decanting, may be utilized as determined by one of skill in the art.

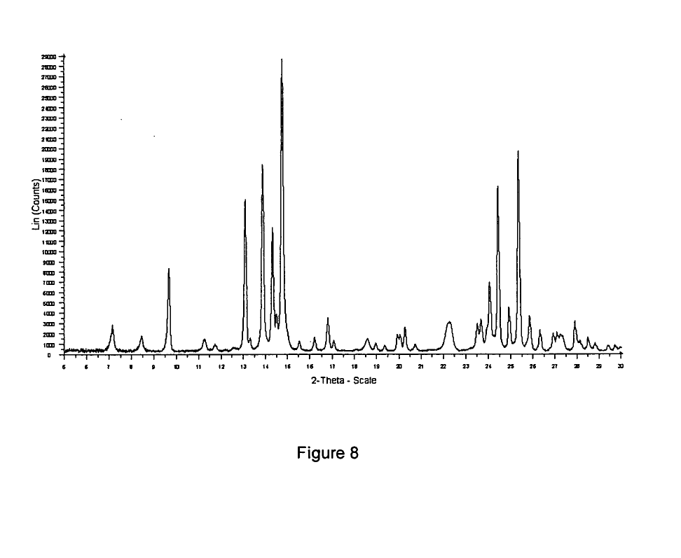

The powder XRD pattern of solvate Form I described herein was obtained using X-ray crystallographic techniques known to those of skill in the art. See, FIG. 8. In one embodiment, the XRD pattern of solvate Form I includes multiple peaks that differ from the XRD peaks obtained for polymorph Forms I, II, and III, solvate Forms II, III, and IV, and the hydrate. In another embodiment, the XRD pattern for solvate Form I contains one large peak and several smaller peaks. The XRD for solvate Form I includes a peak at 2θ of about 14.8°±0.3° at greater than about 95% relative intensity. Desirably, the peak at 2θ of about 14.8°±0.3° is at about 100% relative intensity. The XRD for solvate Form I may also include peaks at 2θ of about 13.1°±0.3°, 13.9°±0.3°, 14.3°±0.3°, 24.5°±0.3°, or 25.4±0.3° at varying intensities. Desirably, the XRD for solvate Form I lacks the peak for polymorph Form I at about 18.5°.

Other techniques can be utilized to characterize solvate Form I and include DSC and NMR. In one embodiment, DSC techniques can be utilized to distinguish solvate Form I of 5-(4′-fluoro-2′-oxo-1′,2′-dihydrospiro[cyclopropane-1,3′-indoil]-5′-yl)-1-methyl-1H-pyrrole-2-carbonitrile from polymorph Form I. In another embodiment, solution NMR may be used to verify the purity and presence of solvate Form I of 5-(4′-fluoro-2′-oxo-1′,2′-dihydrospiro[cyciopropanze-1,3-indol]-5′-yl)-1-methyl-1H-pyrroie-2-carbonitrile and specifically the presence of DMF in the molecule.

### E. Solvate Form II of 5-(4′-fluoro-2′-oxo-1′,2′-dihydrospiro[cyclopropane-1,3′-indol]-5′-yl)-1-methyl-1H-pyrrole-2-carbonitrile

A second novel solvate of 5-(4′-fluoro-2′-oxo-1′,2′-dihydrospiro[cyclopropane-1,3′-indol]-5′-yl)-1-methyl-1H-pyrrole-2-carbonitrile, i.e., solvate Form II of 5-(4′-fluoro-2′-oxo-1′,2′-dihydrospiro[cyclopropane-1,3′-indol]-5′-yl)-1-methyl-1H-pyrrole-2-carbonitrile, is described herein. Solvate Form II has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and the novel polymorph Forms II and III, solvate Forms I, III, and IV, and the hydrate described herein. Specifically, as evidenced by spectroscopic techniques, solvate Form II contains dimethylformamide in the crystal lattice, which differs from solvate Form I.

In one embodiment, solvate Form II of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. Specifically, solvate Form II of 5-(4′-fluoro-2′-oxo-1′,2′-dihydrospiro[cyclopropane-1,3′-indol]-5′-yl)-1-methyl-1H-pyrrole-2-carbonitrile is prepared by dissolving the hydrate of 5-(4′-fluoro-2′-oxo-1′,2′-dihydrospiro[cyclopropane-1,3′-indol]-5′-yl)-1-methyl-1H-pyrrole-2-carbonitrile in DMF. The DMF solution is then heated to about 40° C. to about 60° C. In one embodiment, the DMF solution is then heated to about 45 to about 55° C. The DMF is then evaporated from the solution using reduced pressures, e.g., a full vacuum. Solvate Form II may then be collected using techniques known to those of skill in the art.

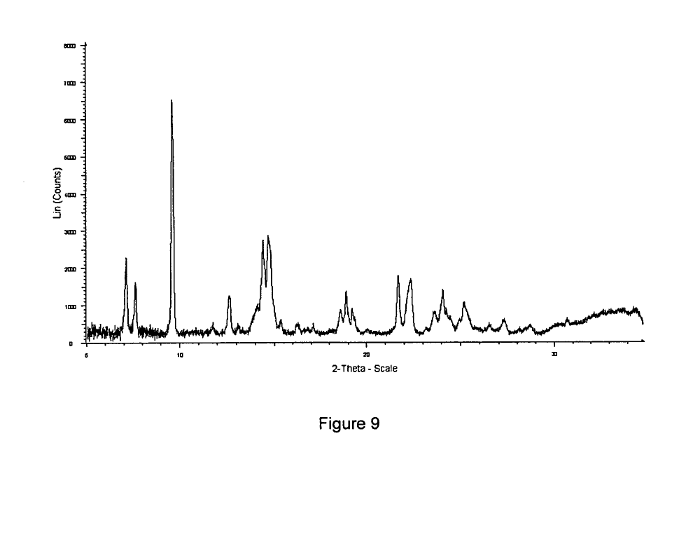

The powder XRD pattern of solvate Form II described herein was obtained using X-ray crystallographic techniques known to those of skill in the art. See, FIG. 9. In one embodiment, the XRD pattern of solvate Form II includes multiple peaks that differ from the XRD peaks obtained for polymorph Forms I, II, and III, solvate Forms 1, III, and IV, and the hydrate. In another embodiment, the XRD pattern for solvate Form II contains one large peak and several smaller peaks. The XRD for solvate Form II includes a peak at 2θ of about 9.5°±0.3° at greater than about 95% relative intensity. Desirably, the peak at 2θ of about 9.5°±0.3° is at about 100% relative intensity. The XRD for solvate Form II may also include peaks at 2θ of about 7.6°±0.3°, 14.7°±0.3°, 21.7°±0.3°, or 22.3±0.3° at varying intensities. Desirably, the XRD for solvate Form II lacks the peak for polymorph Form I at about 18.5°.

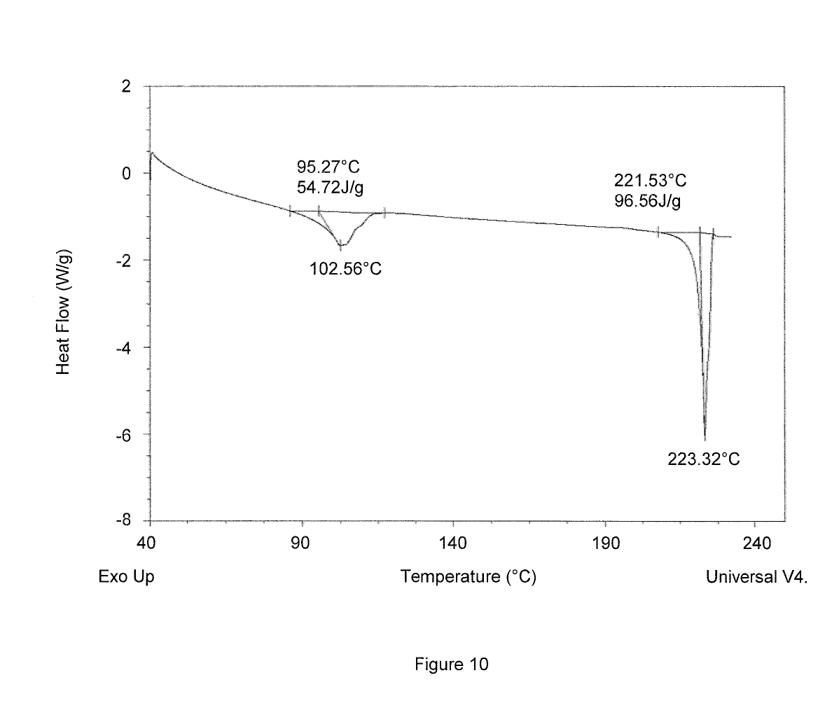

DSC techniques can also be utilized to distinguish solvate Form II of 5-(4′-fluoro-2′-oxo-1′,2′-dihydrospiro[cyclopropane-1,3′-indol]-5′-yl)-1-methyl-1H-pyrrole-2-carbonitrile from polymorph Forms I, II, and III and solvate Form I. See, FIG. 10. In one embodiment, the DSC thermogram of solvate Form II differs from the DSC thermogram of polymorph Form I and includes an endothermic peak with a Tₒₙₛₑₜ of about 221.5° C.±1° C. In another embodiment, the DSC thermogram of solvate Form II differs from the DSC thermogram of polymorph Form I and includes an endothermic peak with a Tₒₙₛₑₜ of about 95.3° C.±1° C.

Solution NMR can also be used to verify the purity and presence of solvate Form II of 5-(4′-fluoro-2′-oxo-1′,2′-dihydrospiro[cyclopropane-1,3′-indol]-5′-yl)-1-methyl-1H-pyrrole-2-carbonitrile and the presence of the DMF in the lattice.

### F. Solvate Form III of 5-(4′-fluoro-2′-oxo-1′,2′-dihydrospiro[cyclopropane-1,3′-indol]-5′-yl)-1-methyl-1H-pyrrole-2-carbonitrile

A third novel solvate form of 5-(4′-fluoro-2′-oxo-1′,2′-dihydrospiro[cyclopropane-1,3-indol]-5′-yl)-1-methyl-1H-pyrrole-2-carbonitrile is provided and designated as solvate Form III. Solvate Form III has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and the novel polymorph Forms II and III, solvate Forms I, II, and IV, and the hydrate described herein As evidenced by spectroscopic techniques, solvate Form III contains toluene in the crystal lattice.

Also provided are methods for preparing solvate Form III of 5-(4′-fluoro-2′-oxo-1,2′-dihydro spiro[cyclopropane-1,3′-indol]-5′-yl)-1-methyl-1,1-pyrrole-2-carbonitrile. In one embodiment, solvate Form III of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. Specifically, solvate Form III of 5-(4′-fluoro-2′-oxo-1′,2′-dihydrospiro[cyclopropane-1,3′-indol]-5′-yl)-1-methyl-1H-pyrrole-2-carbonitrile is prepared by slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in toluene. In one embodiment, the hydrate is slurried in toluene for at least about 60 hours. In another embodiment, the hydrate is slurried in toluene for about 60 hours to about 7 days. Solvate Form III may then be isolated from the solution using filtration. However, other techniques may be utilized as determined by one of skill in the art.

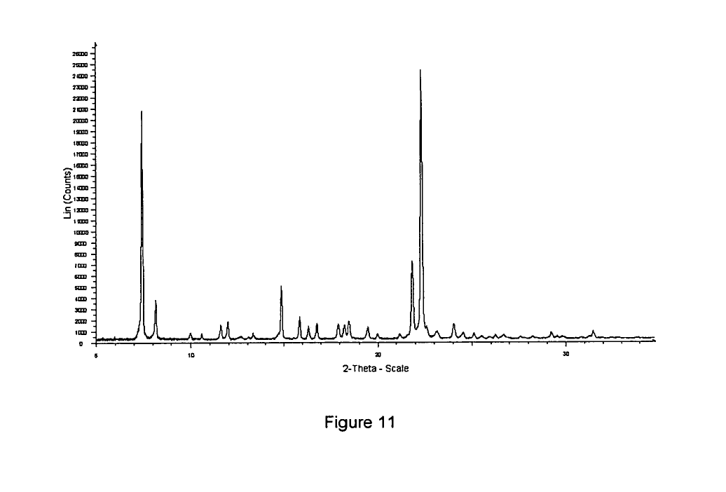

The powder XRD pattern of solvate Form III described herein was obtained using X-ray crystallographic techniques known to those of skill in the art. See, FIG. 11. In one embodiment, the XRD pattern of solvate Form III includes multiple peaks that differ from the XRD peaks obtained for polymorph Forms I, II, and III, solvate Forms I, II, and IV, and the hydrate. In another embodiment, the XRD pattern for solvate Form III contains one large peak and several smaller peaks. The XRD for solvate Form III includes a peak at 2θ of about 11.9°±0.30 at greater than about 95% relative intensity. Desirably, the peak at 2θ of about 11.9°±0.30 is at about 100% relative intensity. The XRD for solvate Form III may also include peaks at 2θ of about 7.6°±0.3°, 8.4°±0.3°, 11.6°±0.3°, 13.4°±0.3°, 15.1°±0.3°, 15.7°±0.3°, 16.9°±0.3°, 18.3°±0.3°, 18.6°±0.3°, 19.9°±0.3°, 22.5°±0.3°, 22.8°±0.3°, 25.8°±0.3°, and 26.4°±0.3° at varying intensities. Desirably, the XRD for solvate Form III lacks the peak for polymorph Form I at about 18.5°.

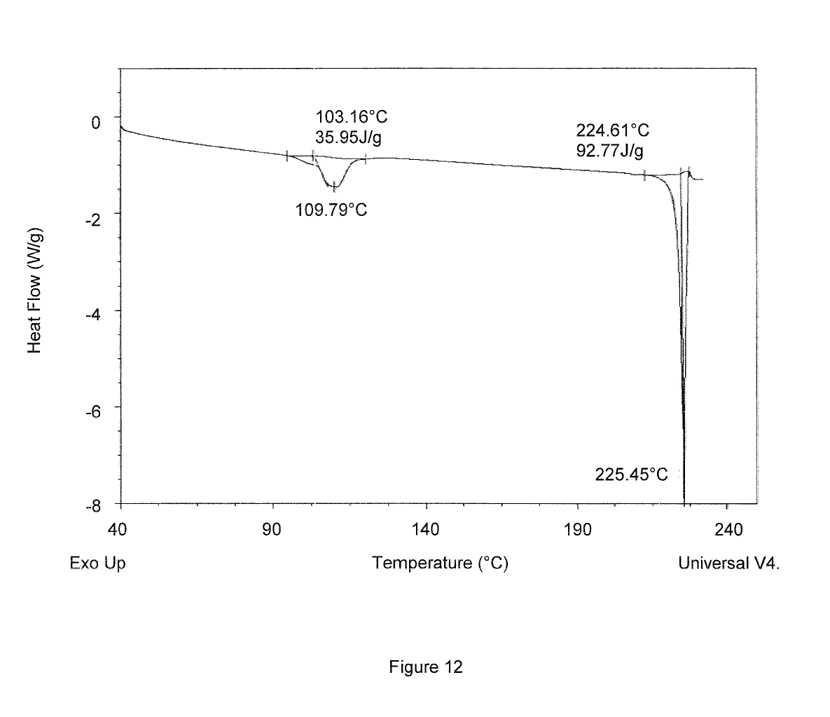

DSC techniques can also be utilized to distinguish solvate Form III of 5-(4′-fluoro-2′-oxo-1′,2′-dihydrospiro[cyclopropane-1,3′-indol]-5′-yl)-1-methyl-1H-pyrrole-2-carbonitrile from polymorph Forms I, II, and III and solvate Forms I and II. See, FIG. 12. In one embodiment, the DSC thermogram of solvate Form III differs from the DSC thermogram of polymorph Form I and includes an endothermic peak with a Tₒₙₛₑₜ of about 224.6° C.±1° C. In another embodiment, the DSC thermogram of solvate Form III differs from the DSC thermogram of polymorph Form I and includes an endothermic peak with a Tₒₙₛₑₜ of about 103.2° C.±1° C.

Solution NMR can also be used to verify the purity and presence of solvate Form III of 5-(4′-fluoro-2′-oxo-1′,2′-dihydrospiro[cyclopropane-1,3′-indol]-5′-yl)-1-methyl-1H-pyrrole-2-carbonitrile and the presence of toluene in the molecule.

### G. Solvate Form IV of 5-(4′-fluoro-2′-oxo-1′,2′-dihydrospiro[cyclopropane-1,3′-indol]-5′-yl)-1-methyl-1H-pyrrole-2-carbonitrile

Another novel solvate of 5-(4′-fluoro-2′-oxo-1′,2′-dihydrospiro[cyclopropane-1,3′-indol]-5′-yl)-1-methyl-1H-pyrrole-2-carbonitrile, denoted herein as solvate Form IV, is described. Solvate Form IV has a different structure of the crystal lattice of 5-(4′-fluoro-2′-oxo-1′,2′-dihydrospiro[cyclopropane-1,3′-indol]-5′-yl)-1-methyl-1H-pyrrole-2-carbonitrile from polymorph Form I described in the art and the novel polymorph Forms II and III, solvate Forms I, II, and III, and the hydrate described herein As evidenced by spectroscopic techniques, solvate Form IV contains heptane in the crystal lattice.

Further provided herein are methods for preparing solvate Form IV of 5-(4′-fluoro-2′-oxo-1′,2′-dihydrospiro[cyclopropane-1,3′-indol]-5′-yl)-1-methyl-1H-pyrrole-2-carbonitrile, i.e., a fourth novel solvate of 5-(4′-fluoro-2′-oxo-1′,2′-dihydrospiro[cyclopropane-1,3′-indol]-5′-yl)-1-methyl-1H-pyrrole-2-carbonitrile. In one embodiment, solvate Form IV of 5-(4′-fluoro-2′-oxo-1′,2′-dihydrospiro[cyclopropane-1,3′-indol]-5′-yl)-1-methyl-1H-pyrrole-2-carbonitrile is prepared from the hydrate of 5-(4′-fluoro-2′-oxo-1′,2′-dihydrospiro[cyclopropane-1,3′-indol]-5′-yl)-1-methyl-1H-pyrrole-2-carbonitrile. Specifically, solvate Form IV of 5-(4′-fluoro-2′-oxo-1′,2′-dihydro spiro[cyclopropane-1,3′-indol]-5′-yl)-1-methyl-1H-pyrrole-2-carbonitrile was prepared by dissolving the hydrate of 5-(4′-fluoro-2′-oxo-1′,2′-dihydrospiro[cyclopropane-1,3′-indol]-5′-yl)-1-methyl-1H-pyrroie-2-carbonitrile in tetrahydrofuran (THF). Heptane is then quickly added to the THF solution. The term “quickly” as used herein describes the rate of addition of the heptane. Desirably, the heptane is poured into the THF solution without stopping. Desirably, the THF solution is stirred during heptane addition. In one embodiment, excess heptane is added to the THF solution. In another embodiment, the volume of THF to heptane is 1:1. The amount of “excess” heptane could readily be determined by one of skill in the art. In one embodiment, the term excess refers to a heptane volume that is 1.5 times or greater the amount of THF. In another embodiment, excess refers to a volume that is 1.5, 2, 3, 4, 5, 6, 7, 8, 9, or 10 times the amount of THF. Solvate Form IV may then be isolated from the solution using filtration. However, other techniques may be utilized as determined by one of skill in the art.

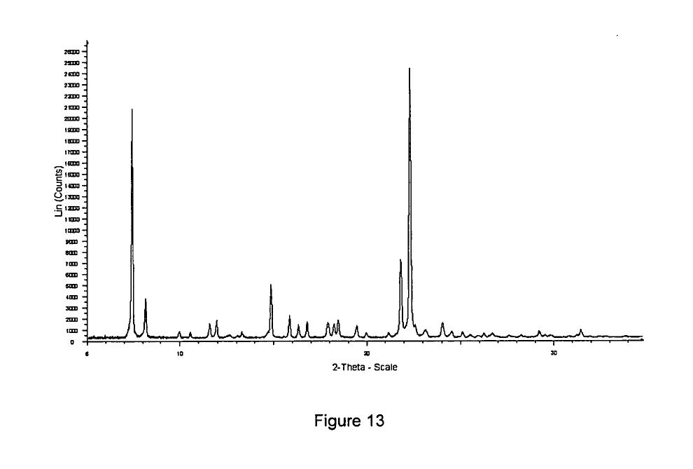

In one embodiment, XRD techniques are utilized to distinguish solvate Form IV from polymorph Forms I, II, and III and solvate Forms I, II, and III. The powder XRD pattern of solvate Form IV described herein was obtained using X-ray crystallographic techniques known to those of skill in the art. See, FIG. 13. In one embodiment, the XRD pattern of solvate Form IV includes multiple peaks that differ from the XRD peaks obtained for polymorph Forms I, II, and III, solvate Forms 1, II, and III, and the hydrate. In another embodiment, the XRD pattern for solvate Form IV contains one large peak and several smaller peaks. The XRD for solvate Form IV includes a peak at 2θ of about 22.3°±0.3° at greater than about 95% relative intensity. Desirably, the peak at 2θ of about 22.3°±0.3° is at about 100% relative intensity. The XRD for solvate Form IV may also include peaks at 2θ of about 7.4°±0.3°, 8.1°±0.3°, 14.8°±0.3°, and 21.8°±0.3° at varying intensities. Desirably, the XRD for solvate Form IV lacks the peak for polymorph Form I at about 18.50.

DSC techniques can also be utilized to distinguish solvate Form IV of 5-(4′-fluoro-2′-oxo-1′,2′-dihydrospiro[cyclopropane-1,3′-indol]-5′-yl)-1-methyl-1H-pyrrole-2-carbonitrile from polymorph Form I.

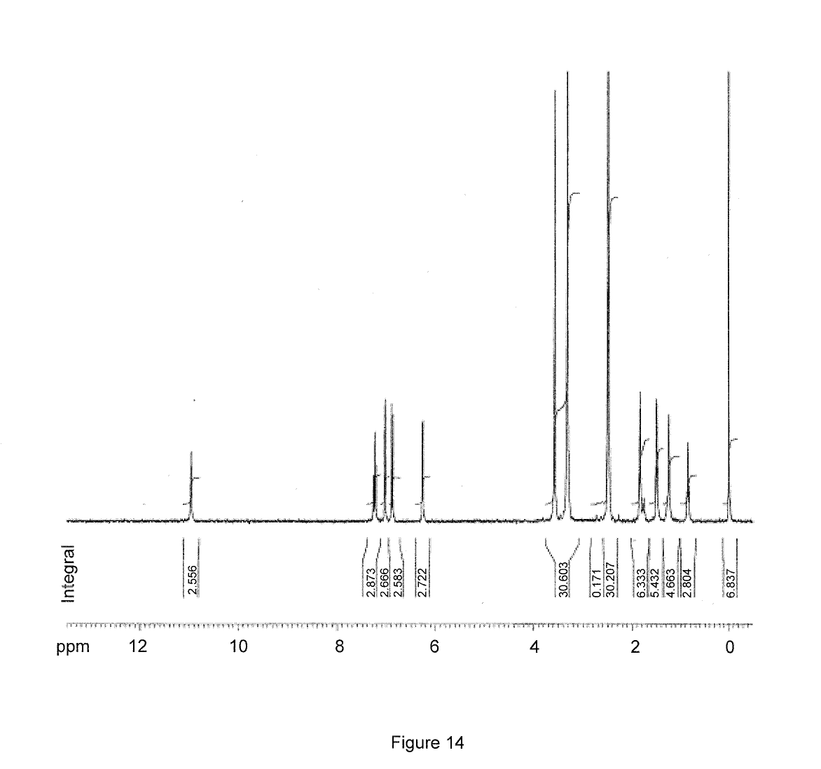

Solution NMR was also used to verify the purity and presence of solvate Form IV of 5-(4′-fluoro-2′-oxo-1′,2′-dihydrospiro[cyclopropane-1,3′-indol]-5′-yl)-1-methyl-1H-pyrrole-2-carbonitrile and the presence of heptane in the lattice. See, FIG. 14. In one embodiment, the solution NMR spectrum for solvate Form IV was identical to the solution NMR spectrum for polymorph Form I with the exception that the NMR spectrum for solvate Form IV also contained peaks that verified the presence of heptane in the crystal lattice. Specifically, the peaks in the NMR spectrum that corresponded to the heptane molecules were at about δ 0.86 and 1.25±0.1 in dimethylsulfoxide (DMSO) solvent. However, one of skill in the art would readily understand that the chemical shift of the peaks in the NMR spectrum will vary depending on the solvent utilized.

### H. Micronized Polymorph Forms II and III, Solvate Forms I, II, III, and IV, and the Hydrate of 5-(4′-fluoro-2′-oxo-1′,2′-dihydrospiro[cylopropane-1,3′-indol]-5′-yl)-1-methyl-1H-pyrrole-2-carbonitrile

Polymorph Form I and novel polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile can be micronized under nitrogen and conventional micronizing techniques, e.g., with a Trost or jet mill as described above for polymorph Form I. Micronized 5-(4′-fluoro-2′-oxo-1′,2′-dihydrospiro[cyclopropane-1,3′-indol]-5′-yl)-1-methyl-1H-pyrrole-2-carbonitrile polymorph Form I has median particle size less than about 8 μm±1 μm.

The different, novel forms, i.e., polymorphs, solvates, and/or hydrate, may be micronized independently or two or more may be combined for micronization In one embodiment, micronized solvate Form I of 5-(4′-fluoro-2′-oxo-1′,2′-dihydrospiro[cyclopropane-1,3′-indol]-5′-yl)-1-methyl-1H-pyrrole-2-carbonitrile Form I has median particle size less than about 10 μm±1 μm. In a further embodiment, micronized solvate Form II of 5-(4′-fluoro-2′-oxo-1′,2′-dihydrospiro[cyclopropane-1,3′-indol]-5′-yl)-1-methyl-1H-pyrrole-2-carbonitrile has median particle size less than about 10 μm±1 μm. In another embodiment, micronized solvate Form III of 5-(4′-fluoro-2′-oxo=1′,2′-dihydro spiro[cyclopropane-1,3′-indol]-5′-yl)-1-methyl-1H-pyrrole-2-carbonitrile has median particle size less than about 10 μm±1 μm. In yet another embodiment, micronized solvate Form IV of 5-(4′-fluoro-2′-oxo-1′,2′-dihydrospiro[cyclopropane-1,3′-indol]-5′-yl)-1-methyl-1H-pyrrole-2-carbonitrile has median particle size less than about

In still another embodiment, samples of the hydrate are micronized under 60 psi (4 bar) nitrogen. In one embodiment, the median particle size of a sample of micronized hydrate of 5-(4′-fluoro-2′-oxo-1′,2′-dihydrospiro[cyclopropane-1,3′-indol]-5′-yl)-1-methyl-1H-pyrrole-2-carbonitrile is 90% less than about 10 μm.

### I. Compositions Containing Polymorph Forms II and/or III, Solvate Forms I, II, III, and/or IV, and/or the Hydrate of 5-(4′-fluoro-2′-oxo-1′,2′-dihydrospiro[cyclopropane-1,3′-indol]-5′-yl)-1-methyl-1H-pyrrole-2-carbonitrile

Also provided are compositions, desirably pharmaceutical compositions, containing one or more of polymorph Form II or Form II, solvate Form I, II, III, or IV, and the hydrate of 5-(4′-fluoro-2′-oxo-1′,2′-dihydrospiro[cyclopropane-1,3′-indol]-5′-yl)-1-methyl-1H-pyrrole-2-carbonitrile alone or in combination with polymorph Form I. In one embodiment, a pharmaceutical composition containing one or more of polymorph Form II or III, solvate Form I, II, III, or IV, or hydate of 5-(4′-fluoro-2′-oxo-1′,2′-dihydrospiro[cyclopropane-1,3′-indol]-5′-yl)-1-methyl-1H-pyrrole-2-carbonitrile and a pharmaceutically acceptable carrier is provided.

The compositions typically contain a pharmaceutically acceptable carrier, but can also contain other suitable components. Typically, the additional components are inert and do not interfere with the function of the required components of the compositions. The compositions can further include other adjuvants, syrups, elixirs, diluents, binders, lubricants, surfactants, granulating agents, disintegrating agents, emollients, metal chelators, pH adjustors, surfactants, fillers, disintegrants, and combinations thereof, among others.

Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA).

Binders can include, without limitation, povidone, cellulose, methylcellulose, hydroxymethylcellulose, carboxymethylcellulose calcium, carboxymethylcellulose sodium, hydroxypropylcellulose, hydroxypropylmethylcellulose phthalate, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone.

Lubricants can include light anhydrous silicic acid, talc, stearic acid, sodium lauryl sulfate, magnesium stearate and sodium stearyl fumarate, among others. In one embodiment, the lubricant is magnesium stearate.

Granulating agents can include, without limitation, silicon dioxide, starch, calcium carbonate, pectin, crospovidone, and polyplasdone, among others.

Disintegrating agents or disintegrants can include starch, carboxymethylcellulose, substituted hydroxypropylcellulose, sodium bicarbonate, calcium phosphate, calcium citrate, sodium starch glycolate, pregelatinized starch or crospovidone, among others.

Emollients can include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Surfactants can include polysorbates, sorbitan esters, poloxamer, or sodium lauryl sulfate. In one embodiment, the surfactant is sodium lauryl sulfate.

Metal chelators can include physiologically acceptable chelating agents including edetic acid, malic acid, or fumaric acid. In one embodiment, the metal chelator is edetic acid.

pH adjusters can also be utilized to adjust the pH of a solution containing one or more of polymorph Form II or III, solvate Form I, II, III, or IV, and the hydrate of 5-(4′-fluoro-2′-oxo-1′,2′-dihydrospiro[cyclopropane-1,3′-indol]-5′-yl)-1-methyl-1H-pyrrole-2-carbonitrile, optionally containing polymorph Form I, to about 4, about 5, or about 6. In one embodiment, the pH of a solution containing polymorph Form IT or III, solvate Form I, II, III, or IV, and the hydrate of 5-(4′-fluoro-2′-oxo-1′,2′-dihydrospiro[cyclopropane-1,3′-indol]-5′-yl)-1-methyl-1H-pyrrole-2-carbonitrile, optionally containing polymorph Form I, is adjusted to a pH of about 4.6. pH adjustors can include physiologically acceptable agents including citric acid, ascorbic acid, fumaric acid, or malic acid, and salts thereof. In one embodiment, the pH adjuster is citric acid.

Additional fillers that can be used in the composition include mannitol, calcium phosphate, pregelatinized starch, or sucrose.

### J. Methods of Using Polymorph Forms II and/or III, Solvate Forms I, II, III, and/or IV, and/or the Hydrate of 5-(4′-fluoro-2′-oxo-1′,2′-dihydrospiro[cyclopropane-1,3′-indol]-5′-yl)-1-methyl-1H-pyrrole-2-carbonitrile

Further provided are methods of delivering polymorph Forms II and/or III, solvate Forms I, II, III, and/or IV, and/or the hydrate of 5-(4′-fluoro-2′-oxo-1′,2′-dihydrospiro[cyclopropane-1,3′-indol]-5′-yl)-1-methyl-1H-pyrrole-2-carbonitrile to a patient, where the method includes administering polymorph Form II and/or III, and/or solvate Form I, TI, III, and/or IV, and/or the hydrate.

The dosage requirements of polymorph Form II or III, solvate Form I, II, III, or IV, or the hydrate may vary based on the severity of the symptoms presented and the particular subject being treated. Treatment can be initiated with small dosages less than the optimum dose of polymorph Form II or III, solvate Form I, II, III, or IV, or the hydrate. Thereafter the dosage is increased until the optimum effect under the circumstances is reached. Precise dosages will be determined by the administering physician based on experience with the individual subject treated. In general, polymorph Form II or III, solvate Form I, II, III, or IV, or the hydrate is most desirably administered at a concentration that will generally afford effective results without causing any unacceptable harmful or deleterious side effects. For example, an effective amount of polymorph Form TI or III, solvate Form I, II, III, or IV, or the hydrate is generally, e.g., about 0.05 mg to about 1 mg, about 0.05 mg to about 0.3 mg, about 0.05 mg, about 0.075 mg, about 0.1 mg, about 0.15 mg, about 0.2 mg, or about 0.3 mg.

Polymorph Form II and/or III, and/or solvate Form I, II, III, and/or IV, and/or the hydrate is therefore useful in contraception and hormone replacement therapy. Polymorph Form II and/or III, solvate Form I, II, III, and/or IV, and/or the hydrate is also useful in the treatment and/or prevention of fibroids, specifically uterine fibroids; benign prostatic hypertrophy; benign and malignant neoplastic disease; dysfunctional bleeding; uterine leiomyomata; endometriosis; polycystic ovary syndrome; and hormone-dependent carcinomas and adenocarcinomas of the pituitary, endometrium, kidney, uterine, ovary, breast, colon, and prostate and other hormone-dependent tumors; and treating symptoms of premenstrual syndrome and premenstrual dysphoric disorder. Additional uses of one or more of polymorph Form II or III, solvate Form I, II, III, or IV, or the hydrate include stimulation of food intake or the synchronization of estrus.

One or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile can be formulated in any form suitable for the desired route of delivery using a pharmaceutically effective amount of polymorph Form II or III, solvate Form I, II, III, or IV, or the hydrate, optionally in combination with polymorph Form I. For example, polymorph Form II or III, solvate Form I, II, III, or IV, or the hydrate can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release. Desirably, delivery is oral.

For example, one or more of polymorph Form II or III, solvate Form I, II, III, or IV, or the hydrate, optionally in combination with polymorph Form I, may be formulated for administration orally in such forms as tablets, capsules, microcapsules, dispersible powders, granules, or suspensions containing, e.g., about 0.05 to 5% of suspending agent, syrups containing, e.g., about 10 to 50% of sugar, and elixirs containing, e.g., about 2θ to 50% ethanol, and the like. The preferred pharmaceutical compositions from the standpoint of ease of preparation and administration are solid compositions, particularly tablets and hard-filled or liquid-filled capsules.

One of more of polymorph Form II or III, solvate Form I, II, III, or IV, or the hydrate, optionally in combination with polymorph Form I, may also be administered parenterally or intraperitoneally. Solutions or suspensions of polymorph Form II or III, solvate Form I, II, III, or IV, or the hydrate can be prepared in water suitably mixed with a surfactant such as hydroxypropylcellulose. Dispersions can also be prepared in glycerol, liquid, polyethylene glycols and mixtures thereof in oils. Under ordinary conditions of storage and use, these preparations contain a preservative to prevent the growth of microorganisms. Typically, such sterile injectable solutions or suspensions contain about 0.05 to 5% suspending agent in an isotonic medium. Such pharmaceutical preparations may contain, e.g., about 25 to about 90% of the active ingredient in combination with the carrier, more usually between about 5% and 60% by weight.

In another embodiment, one or more of polymorph Form II or III, solvate Form I, II, III, or IV, or the hydrate, optionally in combination with polymorph Form I, is delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile, stable under conditions of manufacture and storage, and free of the contaminating action of microorganisms such as bacteria and fungi.

The carrier can be a solvent or dispersion medium containing, e.g., water, ethanol (e.g., glycerol, propylene glycol and liquid polyethylene glycol), oils, and mixtures thereof. Desirably the liquid carrier is water. In one embodiment, the oil is vegetable oil. Optionally, the liquid carrier contains a suspending agent. In another embodiment, the liquid carrier is an isotonic medium and contains 0.05 to about 5% suspending agent.

In a further embodiment, one or more of polymorph Form II or III, solvate Form I, II, III, or IV, or the hydrate, optionally in combination with polymorph Form I, is delivered rectally in the form of a conventional suppository.

In another embodiment, one or more of polymorph Form II or III, solvate Form I, II, III, or IV, or the hydrate, optionally in combination with polymorph Form I, is delivered vaginally in the form of a conventional suppository, cream, gel, ring, or coated intrauterine device (IUD).

In yet another embodiment, one or more of polymorph Form II or III, solvate Form I, II, III, or IV, or the hydrate, optionally in combination with polymorph Form I, is delivered intranasally or intrabronchially in the form of an aerosol.

In a further embodiment, one or more of polymorph Form II or III, solvate Form I, II, III, or IV, or the hydrate, optionally in combination with polymorph Form I, is delivered transdermally or by sustained release through the use of a transdermal patch containing polymorph Form II or III, solvate Form I, II, III, or IV, or the hydrate and an optional carrier that is inert to polymorph Form II or III, solvate Form I, II, III, or IV, or the hydrate, is nontoxic to the skin, and allows for delivery of polymorph Form II or III, solvate Form I, II, III, or IV, or the hydrate for systemic absorption into the blood stream. Such a carrier can be a cream, ointment, paste, gel, or occlusive device. The creams and ointments can be viscous liquid or semisolid emulsions. Pastes include absorptive powders dispersed in petroleum or hydrophilic petroleum. Further, a variety of occlusive devices can be utilized to release polymorph Form II or III, solvate Form I, II, III, or IV, or the hydrate into the blood stream and include semi-permeable membranes covering a reservoir containing the active reagents, or a matrix containing the active reagents.

The use of sustained delivery devices can be desirable, in order to avoid the necessity for the patient to take medications on a daily basis. The term “sustained delivery” is used herein to refer to delaying the release of an active agent, i.e., one or more polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile, optionally in combination with polymorph Form I, until after placement in a delivery environment, followed by a sustained release of the agent at a later time. A number of sustained delivery devices are known in the art and include hydrogels (U.S. Pat. Nos. 5,266,325; 4,959,217; 5,292,515); osmotic pumps (U.S. Pat. Nos. 4,295,987 and 5,273,752 and European Patent No. 314,206, among others); hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devices composed of, e.g., polyesters, polyanhydrides, or lactic acid/glycolic acid copolymers (U.S. Pat. No. 5,817,343). For use in such sustained delivery devices, one or more of polymorph Form II or III, solvate Form I, II, III, or IV, or the hydrate, optionally in combination with polymorph Form I, can be formulated as described herein. See, U.S. Pat. Nos. 3,845,770; 3,916,899; 3,536,809; 3,598,123; and 4,008,719.

Desirably, one or more of polymorph Form II or III, solvate Form I, II, III, or IV, or the hydrate, optionally in combination with polymorph Form I, is formed into a suitable dosing unit for delivery to a patient. Suitable dosing units include oral dosing units, such as a directly compressible tablets, capsules, powders, suspensions, microcapsules, dispersible powders, granules, syrups, elixirs, and aerosols. In one embodiment, one or more of polymorph Form II or III, solvate Form I, II, III, or IV, or the hydrate, optionally in combination with polymorph Form I, is compressed into a tablet, which is optionally added to a capsule, or polymorph Form II or III, solvate Form I, II, III, or IV, or the hydrate, optionally in combination with polymorph Form I, is added directly to a capsule. Polymorph Form II or III, solvate Form I, II, III, or IV, or the hydrate can also be formulated for delivery by other suitable routes. These dosing units are readily prepared using the methods described herein and those of skill in the art.

Solid forms, including tablets, caplets, and capsules containing polymorph Form II or III, solvate Form I, II III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile can be formed by dry blending one or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile, optionally in combination with polymorph Form I, with the components described above. In one embodiment, the capsules include hydroxypropyl methylcellulose, hypromellose capsule, or a hard shell gelatin capsule. The tablets or caplets that contain one or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile, optionally in combination with polymorph Form I, are optionally film-coated. Suitable film-coatings are known to those of skill in the art. For example, the film-coating can be selected from among polymers such as hydroxypropylmethylcellulose, ethyl cellulose, polyvinyl alcohol, and combinations thereof.

A pharmaceutically effective amount of polymorph Form II or III, solvate Form I, II, III, or IV, or the hydrate can vary depending on the other components of the composition being delivered, mode of delivery, severity of the condition being treated, the patient's age and weight, and any other active ingredients used in the composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

It is contemplated that when polymorph Form II or III, solvate Form I, II, III, or IV, or the hydrate is used for contraception or hormone replacement therapy, it can be administered in conjunction with one or more other progesterone receptor agonists, estrogen receptor agonists, progesterone receptor antagonists, and selective estrogen receptor modulators, among others.

When utilized for treating neoplastic disease, carcinomas, and adenocarcinomas, polymorph Form II or III, solvate Form I, II, III, or IV, or the hydrate can be administered in conjunction with one or more chemotherapeutic agents which can readily be selected by one of skill in the art.

In one embodiment, a method of preparing a pharmaceutical composition containing one or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile, optionally in combination with polymorph Form I, is described and includes, respectively, combining polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile and one or more of a metal chelator, a pH adjuster, a surfactant, at least one filler, a binder, a disintegrant, and a lubricant.

### K. Kits Containing Polymorph Forms II and/or III, Solvate Forms I, II, III, and/or IV, and/or the Hydrate of 5-(4′-fluoro-2′-oxo-1′,2′-dihydrospiro[cyclopropane-1,3′-indol]-5′-yl)-1-methyl-1H-pyrrole-2-carbonitrile

Also provided are kits or packages containing one or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile. Kits can include one or more of polymorph Form II or III, solvate Form I, II, III, or IV, or the hydrate or in combination with polymorph Form I and a carrier suitable for administration to a mammalian subject as discussed above. Typically, the tablets or capsules are packaged in blister packs, and desirably Ultrx™ 2000 blister packs. In one embodiment, a kit is provided and contains one or more of polymorph Form II or III, solvate Form I, II, III, or IV, or the hydrate of 5-(4′-fluoro-2′-oxo-1′,2′-dihydrospiro[cyclopropane-1,3′-indol]-5′-yl)-1-methyl-1H-pyrrole-2-carbonitrile; and a carrier suitable for administration to a mammalian subject is described.

The kits or packages containing one or more of polymorph Form II or III, solvate Form I, II, III, or IV, or the hydrate are designed for use in the regimens described herein. These kits are desirably designed for daily oral delivery over 21-day, 28-day, 30-day, or 31-day cycles, among others, and more desirably for one oral delivery per day. When polymorph Form II or III, solvate Form I, II, III, or IV, or the hydrate is to be delivered continuously, a package or kit can include one or more of polymorph Form II or III, solvate Form I, II, III, or IV, or the hydrate in each tablet. When polymorph Form II or II, solvate Form I, II, III, or IV, or the hydrate is to be delivered with periodic discontinuation, a package or kit can include placebos on those days when polymorph Form II or III, solvate Form I, II, III, or IV, or the hydrate is not delivered.

Additional components may be co-administered with polymorph Form II or III, solvate Form I, II, III, or IV, or the hydrate and include progestational agents, estrogens, and selective estrogen receptor modulators.

The kits are also desirably organized to indicate a single oral formulation or combination of oral formulations to be taken on each day of the cycle, desirably including oral tablets to be taken on each of the days specified. More desirably, one oral tablet will contain each of the combined daily dosages indicated.

In one embodiment, a kit can include a single phase of a daily dosage of one or more of polymorph Form II or III, solvate Form I, II, III, or IV, or the hydrate, optionally in combination with polymorph Form I, over a 21-day, 28-day, 30-day, or 31-day cycle. Alternatively, a kit can include a single phase of a daily dosage of polymorph Form II or III, solvate Form I, II, III, or IV, or the hydrate over the first 21 days of a 28-day, 30-day, or 31-day cycle. A kit can also include a single phase of a daily dosage of polymorph Form II or III, solvate Form I, II, III, or IV, or the hydrate over the first 28 days of a 30-day or 31-day cycle.

In a further embodiment, a kit can include a single combined phase of a daily dosage of one or more of polymorph Form II or III, solvate Form I, II, III, or IV, or the hydrate, optionally in combination with polymorph Form I, and a progestational agent over a 21-day, 28-day, 30-day, or 31-day cycle. Alternatively, a kit can include a single combined phase of a daily dosage of polymorph Form II or III, solvate Form I, II, III, or IV, or the hydrate and a progestational agent over the first 21 days of a 28-day 30-day, or 31-day cycle. A kit can also include a single combined phase of a daily dosage of polymorph Form II or III, solvate Form I, II, III, or IV, or the hydrate and a progestational agent over the first 28 days of a 30-day or 31-day cycle.

In another embodiment, a 28-day kit can include a first phase of 14 to 28 daily dosage units of one or more of polymorph Form II or III, solvate Form I, II, III, or IV, or the hydrate, optionally in combination with polymorph Form T; a second phase of 1 to 11 daily dosage units of a progestational agent; and, optionally, a third phase of an orally and pharmaceutically acceptable placebo for the remaining days of the cycle.

In yet a further embodiment, a 28-day kit can include a first phase of 14 to 21 daily dosage units of one or more of polymorph Form II or III, solvate Form I, II, III, or IV, or the hydrate, optionally in combination with polymorph Form I; a second phase of 1 to 11 daily dosage units of a progestational agent; and, optionally, a third phase of an orally and pharmaceutically acceptable placebo for the remaining days of the cycle.

In another embodiment, a 28-day kit can include a first phase of 18 to 21 daily dosage units of one or more of polymorph Form II or III, solvate Form I, II, III, or IV, or the hydrate, optionally in combination with polymorph Form I; a second phase of 1 to 7 daily dose units of a progestational agent; and, optionally, an orally and pharmaceutically acceptable placebo for each of the remaining 0 to 9 days in the 28-day cycle.

In yet a further embodiment, a 28-day kit can include a first phase of 21 daily dosage units of one or more of polymorph Form II or III, solvate Form I, II, III, or IV, or the hydrate, optionally in combination with polymorph Form J; a second phase of 3 daily dosage units for days 22 to 24 of a progestational agent; and, optionally, a third phase of 4 daily units of an orally and pharmaceutically acceptable placebo for each of days 25 to 28.

In another embodiment, a 28-day kit can include a first phase of from 14 to 21 daily dosage units of a progestational agent equal in progestational activity to about 35 to about 150 μg levonorgestrel, a second phase of from 1 to 11 daily dosage units of one or more of polymorph Form II or III, solvate Form I, II, III, or IV, or the hydrate, optionally in combination with polymorph Form i; and optionally, a third phase of an orally and pharmaceutically acceptable placebo for the remaining days of the cycle in which no antiprogestin, progestin or estrogen is administered.

In a further embodiment, a 28-day kit can Include a first phase of 14 to 21 daily dosage units of a progestational agent equal in progestational activity to about 35 to about 100 μg levonorgestrel; a second phase of 1 to 11 daily dosage units of one or more of polymorph Form II or III, solvate Form I, II, III, or IV, or the hydrate, optionally in combination with polymorph Form I; and optionally, a third phase of an orally and pharmaceutically acceptable placebo for the remaining days of the cycle in which no antiprogestin, progestin or estrogen is administered.

Desirably, the daily dosage of one or more of polymorph Form II or III, solvate Form I, II, III, or IV, or the hydrate, optionally in combination with polymorph Form I, remains fixed in each particular phase in which it is delivered. It is further desirable that the daily dose units described are to be delivered in the order described, with the first phase followed in order by the second and third phases. To help facilitate compliance with each regimen, it is also preferred that the kits contain the placebo described for the final days of the cycle.

A number of packages or kits are known in the art for use in dispensing pharmaceutical agents for oral use. Desirably, the package has indicators for each day of the 28-day cycle, and more desirably is a labeled blister package, dial dispenser package, or bottle.

The kit can further contain instructions for administering one or more of polymorph Form II or III, solvate Form I, II, III, or IV, or the hydrate.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLES

Thermogravimetric analysis (TGA) data was collected using a TGA SDTA® 851e instrument (Mettler Toledo) under the following parameters:
purge gas (N₂): 40 mL/minute;
scan range: 30 to 250° C.;
scan rate: 10° C./minute.

X-Ray diffraction data was acquired using a D8 ADVANCE® X-ray powder diffractometer (Bruker) having the following parameters:
voltage: 40 kV;
current: 40.0 mA;
scan range (20): 5 to 35°;
scan step size: 0.01°;
total scan time: 33 minutes;
detector: VANTEC™ detector; and
antiscattering slit: 1 mm.

NMR spectra were acquired on a BRUKER® 300 MHz NMR spectrometer using a deuterated solvent and tetramethylsilane (TMS) as an internal standard (δ=0.0 ppm). All δ values are expressed in units of parts per million (ppm).

### Example 1

### Preparation of the hydrate of 5-(4′-fluoro-2′-oxo-1′,2′-dihydrospiro[cyclopropane-1,3′-indol]-5′-yl)-1-methyl-1H-pyrrole-2-carbonitrile

Polymorph Form I of 5-(4′-fluoro-2′-oxo-1′,2′-dihydrospiro[cyclopropane-1,3′-indol]-5′-yl)-1-methyl-1H-pyrrole-2-carbonitrile (11 mg) was slurried in water (0.5 mL) for about 60 hours. The solid present after slurrying was collected and dried for about 15 hours to provide the hydrate of 5-(4′-fluoro-2′-oxo-1′,2′-dihydrospiro[cyclopropane-1,3′-indol]-5′-yl)-1-methyl-1H-pyrrole-2-carbonitrile. The X spectrum, DSC thermogram, and TGA curve of the hydrate were obtained. DSC data were collected using a Q SERIES™ DSC Q1000 (TA instruments) under the following parameters. See, FIGS. 1-3.
purge gas (N₂): 50 mL/minute;
scan range: 40 to 230° C.;
scan rate: 10° C./minute.

### Example 2

### Preparation of Polymorph Form II of 5-(4′-fluoro-2′-oxo-1′,2′-dihydrospiro[cyclopropane-1,3′-indol]-5′-yl)-1-methyl-1H-pyrrole-2-carbonitrile from the hydrate of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrrole-2-carbonitrile (90 mg) was dissolved in tetrahydrofuran (0.63 mL) at about 25° C. for about 60 hours. The solid present in the tetrahydrofuran was then collected to provide polymorph Form II of 5-(4′-fluoro-2′-oxo-1′,2′-dihydrospiro[cyclopropane-1,3′-indol]-5′-yl)-1-methyl-1H-pyrrole-2-carbonitrile.

### Example 3

### Preparation of Polymorph Form II of 5-(4′-fluoro-2′-oxo-1′,2′-dihydrospiro[cyclopropane-1,3′-indol]-5′-yl)-1-methyl-1H-pyrrole-2-carbonitrile from Polymorph Form I of 5-(4′-fluoro-2′-oxo-1′,2′-dihydrospiro[cyclopropane-1,3′-indol]-5′-yl)-1-methyl-1H-pyrrole-2-carbonitrile

Excess polymorph Form I of 5-(4′-fluoro-2′-oxo-1′,2′-dihydrospiro[cyclopropane-1,3′-indol]-5′-yl)-1-methyl-1H-pyrrole-2-carbonitrile was slurried in the solvents set forth in Table 1 at room temperature for about 6 days. The solid was then collected to provide polymorph Form IT of 5-(4′-fluoro-2′-oxo-1′,2′-dihydrospiro[cyclopropane-1,3′-indol]-5′-yl)-1-methyl-1H-pyrrole-2-carbonitrile.

TABLE 1

| Solvent | Amount |
|---|---|
| ethyl acetate | 1 mL |
| methanol | 1 mL |
| isopropyl alcohol | 1 mL |

The XRD spectrum and DSC thermogram of polymorph Form TI were obtained.

DSC data were collected using a Q SERIES™ DSC Q1000 (TA instruments) under the following parameters. See, FIGS. 4-5.
purge gas (N₂): 50 mL/minute;
scan range: 40 to 200° C.;
scan rate: 10° C./minute.

### Example 4

### Preparation of Polymorph Form III of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrrole-2-carbonitrile (44 mg) was dissolved in acetone (1 mL) and the solution heated to about 50° C. for about 0.5 hours. The acetone was then evaporated to provide polymorph Form III of 5-(4′-fluoro-2′-oxo-1′,2′-dihydrospiro[cyclopropane-1,3′-indol]-5′-yl)-1-methyl-1H-pyrrole-2-carbonitrile.

The XRD spectrum and DSC thermogram of polymorph Form III were obtained. DSC data were collected using a Q SERIES™ DSC Q1000 (TA instruments) under the following parameters. See, FIGS. 6-7.
purge gas (N₂): 50 mL/minute;
scan range: 40 to 200° C.;
scan rate: 10° C./minute.

### Example 5

### Preparation of Solvate Form I of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrrole-2-carbonitrile (33 mg) was slurried in dimethylformamide (0.1 mL) at about 25° C. for about 60 hours. The solid present in the dimethylformamide was then collected to provide solvate Form I of 5-(4′-fluoro-2′-oxo-1′,2′-dihydrospiro[cyclopropane-1,3′-indol]-5′-yl)-1-methyl-1H-pyrrole-2-carbonitrile.

The XRD spectrum of solvate Form I was obtained. See, FIG. 8.

### Example 6

### Preparation of Solvate Form II of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrrole-2-carbonitrile (33 mg) was dissolved in dimethylformamide (0.1 mL) at about 50° C. The DMF was then evaporated under reduced pressures to provide solvate Form II of 5-(4′-fluoro-2′-oxo-1′,2′-dihydrospiro[cyclopropane-1,3′-indol]-5′-yl)-1-methyl-1H-pyrrole-2-carbonitrile.

The XRD spectrum and DSC thermogram of solvate Form II were obtained. DSC data were collected using a Q SERIES™ DSC Q1000 (TA instruments) under the following parameters. See, FIGS. 9-10.
purge gas (N₂): 50 mL/minute;
scan range: 40 to 230° C.;
scan rate: 10° C./minute.

### Example 7

### Preparation of Solvate Form III of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrrole-2-carbonitrile (16.8 mg) was slurried in toluene (1 mL) at about 25° C. for about 60 hours. Solvate Form III of 5-(4′-fluoro-2′-oxo-1′,2′-dihydrospiro[cyclopropane-1,3′-indol]-5′-yl)-1-methyl-1H-pyrrole-2-carbonitrile was then collected using filtration.

The XRD spectrum and DSC thermogram of solvate Form III were obtained. DSC data were collected using a Q SERIES™ DSC Q1000 (TA instruments) under the following parameters. See, FIGS. 11-12.
purge gas (N₂): 50 mL/minute;
scan range: 40 to 230° C.;
scan rate: 10° C./minute.

### Example 8

### Preparation of Solvate Form IV of 5-(4′-fluoro-2′-oxo-1′,2′-dihydrospiro[cyclopropane-1,3′-indol]-5′-yl)-1-methyl-1H-pyrrole-2-carbonitrile

The hydrate of 5-(4′-fluoro-2′-oxo-1′,2′-dihydrospiro[cyclopropane-1,3′-indol]-5′-yl)-1-methyl-1H-pyrroie-2-carbonitrile (90 mg) was dissolved in THF (0.9 mL) at about 25° C. An equivolume amount of heptane was quickly added to the THF solution while stirring. Solvate Form IV of 5-(4′-fluoro-2′-oxo-1′,2′-dihydrospiro[cyclopropane-1,3′-indol]-5′-yl)-1-methyl-1H-pyrrole-2-carbonitrile was then collected using filtration.

The XRD and NMR spectra of solvate Form IV were obtained. See, FIGS. 13-14.

All publications cited in this specification are incorporated herein by reference herein. While the invention has been described with reference to a particularly preferred embodiment, it will be appreciated that modifications can be made without departing from the spirit of the invention. Such modifications are intended to fall within the scope of the appended claims.

What is claimed is:

1. A form of 5-(4′-fluoro-2′-oxo-1′,2′-dihydrospiro[cyclopropane-1,3′-indol]-5′-yl)-1-methyl-1H-pyrrole-2-carbonitrile selected from the group consisting of:
(i) polymorph Form II of 5-(4′-fluoro-2′-oxo-1′,2′-dihydrospiro[cyclopropane-1,3′-indol]-5′-yl)-1-methyl-1H-pyrrole-2-carbonitrile comprising:
(a) an X-ray diffraction peak pattern comprising a peak at 2θ of about 12.5° at greater than about 95% relative intensity; and
(b) a differential scanning calorimetry thermogram having an endothermic peak with a Tₒₙₛₑₜ of about 223° C.;
(ii) polymorph Form III of 5-(4′-fluoro-2′-oxo-1′,2′-dihydrospiro[cyclopropane-1,3′-indol]-5′-yl)-1-methyl-1H-pyrrole-2-carbonitrile comprising:
(c) an X-ray diffraction peak pattern comprising a peak at 2θ of about 18.7° at greater than about 95% relative intensity; and
(d) a differential scanning calorimetry thermogram having an endothermic peak with a Tₒₙₛₑₜ of about 220.5° C.;
(iii) solvate Form I of 5-(4′-fluoro-2′-oxo-1′,2′-dihydrospiro[cyclopropane-1,3′-indol]-5′-yl)-1-methyl-1H-pyrrole-2-carbonitrile comprising an X-ray diffraction peak pattern comprising a peak at 2θ of about 14.8° at greater than about 95% relative intensity;
(iv) solvate Form II of 5-(4′-fluoro-2′-oxo-1′,2′-dihydrospiro[cyclopropane-1,3′-indol]-5′-yl)-1-methyl-1H-pyrrole-2-carbonitrile comprising:
(e) an X-ray diffraction peak pattern comprising a peak at 2θ of about 9.5° at greater than about 95% relative intensity; and
(f) a differential scanning calorimetry thermogram having an endothermic peak with a Tₒₙₛₑₜ of about 221.5° C., 95.3° C., or a combination thereof;
(v) solvate Form III of 5-(4′-fluoro-2′-oxo-1′,2′-dihydrospiro[cyclopropane-1,3′-indol]-5′-yl)-1-methyl-1H-pyrrole-2-carbonitrile comprising:
(g) an X-ray diffraction peak pattern comprising a peak at 2θ of about 11.9° at greater than about 95% relative intensity; and
(h) a differential scanning calorimetry thermogram having an endothermic peak with a Tₒₙₛₑₜ of about 224.6° C., 103.2° C., or a combination thereof;
(vi) solvate Form IV of 5-(4′-fluoro-2′-oxo-1′,2′-dihydrospiro[cyclopropane-1,3′-indol]-5′-yl)-1-methyl-1H-pyrrole-2-carbonitrile comprising an X-ray diffraction peak pattern comprising a peak at 2θ of about 22.3° at greater than about 95% relative intensity; and
(vii) hydrate of 5-(4′-fluoro-2′-oxo-1′,2′-dihydrospiro[cyclopropane-1,3′-indol]-5′-yl)-1-methyl-1H-pyrrole-2-carbonitrile comprising:
(j) an X-ray diffraction peak pattern comprising a peak at 2θ of about 8.4° at greater than about 95% relative intensity; and
(k) a differential scanning calorimetry thermogram having one endothermic peak with a Tₒₙₛₑₜ of about 223° C. and a second endothermic peak with a Tₒₙₛₑₜ of about 111° C.

2. The form according to claim 1 which is polymorph Form II and wherein said X-ray diffraction pattern further comprises one or more peaks at 2θ of about 14.1°, 18.9°, and 24.3°.

3. The form according to claim 1, which is polymorph Form II and comprises short, rod-like particles.

4. The form according to claim 1 which is solvate Form I and wherein said X-ray diffraction pattern further comprises one or more peaks at 2θ of about 13.1°, 13.9°, 14.3°, 24.5°, or 25.4°.

5. The form according to claim 1 which is solvate Form I and comprises dimethylformamide.

6. The form according to claim 1 which is solvate Form II and wherein said X-ray diffraction pattern further comprises one or more peaks at 2θ of about 7.6°, 14.7°, 21.7°, and 22.3°.

7. The form according to claim 1 which is solvate Form II and comprises dimethylformamide.

8. The form according to claim 1 which is solvate Form III and wherein said X-ray diffraction pattern further comprises one or more peaks at 2θ of about 7.6°, 8.4°, 11.6°, 13.4°, 15.1°, 15.7°, 16.9°, 18.3°, 18.6°, 19.9°, 22.5°, 22.8°, 25.8°, or 26.4°.

9. The form according to claim 1 which is solvate Form III and comprises toluene.

10. The form according to claim 1 which is solvate Form IV and comprises a nuclear magnetic spectrum comprising peaks at about δ 0.88 and 1.25.

11. The form according to claim 1 which is solvate Form IV and wherein said X-ray diffraction pattern further comprises one or more peaks at 2θ of about 7.4°, 8.1°, 14.8°, and 21.8°.

12. The form according to claim 1 which is solvate Form IV and comprises heptane.

13. The form according to claim 1 which is the hydrate and wherein said X-ray diffraction pattern further comprises a peak at 2θ of about 11.2° and 16.9°.

14. The form according to claim 1 which is the hydrate and comprises thin, plate-like and needle-like particles.

15. The form according to claim 1 which is the hydrate and comprises about 4 to 5% water.

16. The form according to claim 1 which is the hydrate and is hygroscopic.

17. A micronized hydrate of 5-(4′-fluoro-2′-oxo-1′,2′-dihydrospiro[cyclopropane-1,3′-indol]-5′-yl)-1-methyl-1H-pyrrole-2-carbonitrile which has a median particle size less than about 10 μm.

18. A pharmaceutical composition comprising one or more of polymorph Form II, polymorph Form III, solvate Form I, solvate Form II, solvate Form III, solvate Form IV, or the hydrate of 5-(4′-fluoro-2′-oxo-1′,2′-dihydrospiro[cyclopropane-1,3′-indol]-5′-yl)-1-methyl-1H-pyrrole-2-carbonitrile of claim 1 and a pharmaceutically acceptable carrier.

19. A kit comprising one or more of polymorph Form II, polymorph Form III, solvate Form I, solvate Form II, solvate Form III, solvate Form IV, or the hydrate of 5-(4′-fluoro-2′-oxo-1′,2′-dihydrospiro[cyclopropane-1,3′-indol]-5′-yl)-1-methyl-1H-pyrrole-2-carbonitrile of claim 1 and a carrier suitable for administration to a mammalian subject.

20. A method of preparing a pharmaceutical composition comprising one or more of polymorph Form II, polymorph Form III, solvate Form I, solvate Form II, solvate Form III, solvate Form IV, or the hydrate of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising combining one or more of polymorph Form II, polymorph Form III, solvate Form I, solvate Form II, solvate Form III, solvate Form IV, or the hydrate of 5-(4′-fluoro-2′-oxo-1′,2′-dihydrospiro[cyclopropane-1,3′-indol]-5′-yl)-1-methyl-1H-pyrrole-2-carbonitrile and one or more of:
(i) a metal chelator;
(ii) a pH adjuster;
(iii) a surfactant;
(iv) at least one filler;
(v) a binder;
(vi) a disintegrant; and
(vii) a lubricant.

21. A method of contraception; treating or preventing fibroids, uterine leiomyomata, endometriosis, dysfunctional bleeding, polycystic ovary syndrome, or hormone-dependent carcinomas; providing hormone replacement therapy; stimulating food intake; synchronizing estrus; or treating symptoms of premenstrual syndrome and premenstrual dysphoric disorder, said method comprising administering to a female in need thereof one or more of polymorph Form II, polymorph Form III, solvate Form I, solvate Form II, solvate Form III, solvate Form IV, or the hydrate of claim 1.

22. The method according to claim 21, wherein said fibroids are uterine fibroids.

23. The method according to claim 21, wherein said carcinomas are selected from the group consisting of carcinomas of the endometrium, breast, uterine, ovarian and prostate cancer.

24. A method for preparing polymorph Form II of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in ethyl acetate, methanol, or isopropylalcohol at room temperature for about 60 hours.

25. A method for preparing polymorph Form II of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying polymorph Form I of 5-(4′-fluoro-2′-oxo-1′,2′-dihydrospiro[cyclopropane-1,3′-indol]-5′-yl)-1-methyl-1H-pyrrole-2-carbonitrile in ethyl acetate, methanol, or isopropylalcohol at room temperature for about 6 days.

26. A method for preparing polymorph Form III of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising:
(i) dissolving the hydrate of 5-(4′-fluoro-2′-oxo-1′,2′-dihydrospiro[cyclopropane-1,3′-indol]-5′-yl)-1-methyl-1H-pyrrole-2-carbonitrile in acetone;
(ii) heating the product of step (i) to about 50° C.; and
(iii) evaporating said acetone from step (ii).

27. A method for preparing solvate Form I of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in dimethylformamide at about room temperature for at least about 60 hours.

28. The method according to claim 27, wherein the slurrying is performed for more than about 60 hours.

29. A method for preparing solvate Form II of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising:
(i) dissolving the hydrate of 5-(4′-fluoro-2′-oxo-1′,2′-dihydrospiro[cyclopropane-1,3′-indol]-5′-yl)-1-methyl-1H-pyrrole-2-carbonitrile in dimethylformamide;
(ii) heating the product of step (i) to about 50° C.; and
(iii) evaporating said dimethylformamide from step (ii) under vacuum.

30. A method for preparing solvate Form III of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in toluene.

31. The method according to claim 30, wherein said slurrying is performed for more than 60 hours.

32. A method for preparing solvate Form IV of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising:
(i) dissolving the hydrate of 5-(4′-fluoro-2′-oxo-1′,2′-dihydrospiro[cyclopropane-1,3′-indol]-5′-yl)-1-methyl-1H-pyrrole-2-carbonitrile in tetrahydrofuran; and
(ii) adding excess heptane to the product of step (i) quickly with stirring.

33. The method according to claim 32, wherein the tetrahydrofuran to heptane volume ratio is 1:1.

34. A method for preparing the hydrate of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying Form I of 5-(4′-fluoro-2′-oxo-1′,2′-dihydrospiro[cyclopropane-1,3′-indol]-5′-yl)-1-methyl-1H-pyrrole-2-carbonitrile in water at room temperature for more than a week.

35. A method for preparing solvate Form I of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in dimethylformamide at about room temperature for at least about 60 hours.

36. The method according to claim 35, wherein the slurrying is performed for more than about 60 hours.

37. A method for preparing polymorph Form I of 5-(4′-fluoro-2′-oxo-1′,2′-dihydrospiro[cyclopropane-1,3′-indol]-5′-yl)-1-methyl-1H-pyrrole-2-carbonitrile of claim 1, said method comprising slurrying the hydrate of 5-(4′-fluoro-2′-oxo-1′,2′-dihydrospiro[cyclopropane-1,3′-indol]-5′-yl)-1-methyl-1H-pyrrole-2-carbonitrile in ethyl acetate, methanol, or isopropylalcohol at room temperature for about 60 hours.
